(19) United States
(12) Patent Application Publication
(10) Pub. No.: US 2022/0343998 A1
(43) Pub. Date: Oct. 27, 2022

(54) MODELING VARIABILITY IN RADIOSENSITIVITY AND TUMOR IMMUNE CONTEXTURE TO PERSONALIZE RADIOTHERAPY

(71) Applicant: H. LEE MOFFITT CANCER CENTER AND RESEARCH INSTITUTE, INC., Tampa, FL (US)

(72) Inventors: Heiko ENDERLING, Tampa, FL (US); Javier F. TORRES-ROCA, St. Petersburg, FL (US)

(21) Appl. No.: 17/642,758

(22) PCT Filed: Sep. 14, 2020
(86) PCT No.: PCT/US2020/050664

§ 371 Date: Mar. 14, 2022

Related U.S. Application Data
(60) Provisional application No. 62/900,003, filed on Sep. 13, 2019.

(51) Int. Cl. G16B 5/00; A61N 5/10; C12Q 1/6886; G16B 20/00

(52) U.S. Cl. CPC G16B 5/00; A61N 5/1064; C12Q 1/6886; G16B 20/00; C12Q 2600/158; C12Q 2600/156; C12Q 2600/106

(57) ABSTRACT

Disclosed are methods for assessing radiosensitivity based on individual radiation immune sensitivity (iRIS) as a metic to predict radiation response based on its effect on the tumor-immune ecosystem (TIES).

This Application claims the benefit of U.S. Provisional Application No. 62/900,003, filed on Sep. 13, 2019 which is incorporated herein by reference.

### I. BACKGROUND

There is an appreciable spectrum of in vitro sensitivity to ionizing radiation across various cancer cell types that is regulated by the underlying molecular repertoire and the capacity to utilize available nutrients. However, in a tumor comprised of diverse cellular architecture and evolving ecosystems, the response to radiation is much more complex. Despite acknowledged variations in radiosensitivity, the field of radiation oncology currently does not individualize radiation dose prescription based on the intrinsic biology of a patient's tumor. What is needed are new methods for assessing radiosensitivity on a more individualize basis.

### II. SUMMARY

Disclosed are methods related to a systemic multi-tier analysis for determining the radiosentitivity of a tumor and methods of treating a tumor based on this analysis.

In one aspect, disclosed herein are methods of detecting the radiosensitivity of a tumor in a subject comprising obtaining a tumor tissue sample, measuring the individual radiation immune sensitivity (iRIS) phenotype of the tumor tissue sample by measuring the pre-radiation distance of tumor-immune ecosystem (TIES) to the immune mediated tumor elimination (IMTE) region, and the number of effector, suppressor and cancer cells; wherein a iRIS value lower than a median iRIS score (such as, for example, an iRIS score of 3.61) is designated as iRIS^{lo} and indicates that a tumor is radiosensitive and an iRIS value above the median iRIS score (i.e., iRIS^{hi}) indicates that a tumor is radioresistant.

Also disclosed herein are methods of detecting the radiosensitivity of a tumor of any preceding aspect, further comprising measuring radio sensitivity index (RSI) of the tumor tissue sample and identifying the cell populations as RSI^{lo} or RSI^{hi} based on the RSI value compared to a median RSI score; wherein a RSI value lower than a median RSI score (such as, for example 0.42) is designated as RSI^{lo} and indicates that a tumor is radiosensitive and an RSI value above the median RSI score (i.e., RSI^{hi}) indicates that a tumor is radioresistant; and wherein a combined iRIS/RSI phenotype of iRIS^{lo}/RSI^{lo} indicates that the tumor is radiosensitive and wherein a combined phenotype of iRIS^{lo}/RSI^{hi}, iRIS^{hi}/RSI^{lo}, or iRIS^{hi}/RSI^{hi} indicates that eh tumor is radioresistant.

In one aspect, disclosed herein are methods of detecting the radiosensitivity of a tumor of any preceding aspect, further comprising administering fewer fractions of a total radiation dose (such as, for example, less than 20 administered fractions between 1.5 and 2.5 GY for a total administered dose less than 50 GY) to a subject with a tumor that has an iRIS^{lo}/RSI^{lo} phenotype or administering more fractions of a total radiation dose (such as, for example, greater than 20 administered fractions for a total administered dose greater than 30 GY) to a subject with a tumor that has an iRIS^{lo}/RSI^{hi}, iRIS^{hi}/RSI^{lo}, or iRIS^{hi}/RSI^{hi}phenotype.

Also disclosed herein are methods of treating, inhibiting, reducing, decreasing, ameliorating, and/or preventing a cancer and/or metastasis in a subject comprising detecting the radiosensitivity as set forth in any preceding aspect and administering to the subject a total dosage of radiation; wherein the total dosage of radiation is administered over several fractions; and wherein the total number of fractions administered is less than 20 when the iRIS/RSI phenotype is iRIS^{lo}/RSI^{lo}; and wherein the total number of fractions administered is greater than 20 when the iRIS/RSI phenotype is iRIS^{lo}/RSI^{hi}, iRIS^{hi}/RSI^{lo}, or iRIS^{hi}/RSI^{hi}. For example, disclosed herein are methods of treating, inhibiting, reducing, decreasing, ameliorating, and/or preventing a cancer and/or metastasis in a subject comprising obtaining a tumor tissue sample from the subject; measuring the individual radiation immune sensitivity (iRIS), wherein a tumor tissue sample from a cancer that has an iRIS score lower than a median iRIS score is designated as iRIS^{lo}; measuring the radio sensitivity index (RSI) of the tumor tissue sample, wherein a RSI value lower than a median RSI score is designated as RSI^{lo}; and administering to the subject a total dosage of radiation; wherein the total dosage of radiation is administered over several fractions; and wherein the total number of fractions administered is less than 20 when the iRIS/RSI phenotype is iRIS^{lo}/RSI^{lo}; and wherein the total number of fractions administered is greater than 20 when the iRIS/RSI phenotype is iRIS^{lo}/RSI^{hi}, iRIS^{hi}/RSI^{lo}, or iRIS^{hi}/RSI^{hi}. In one aspect, the administered dose of radiation can be between 1.5 and 2.5 GY per fraction.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows tumor types cluster by transcriptional programs. Multidimensional reduction analysis of transcription data by the t-distributed stochastic neighbor embedding (t-SNE) method demonstrates distinct clustering of tumor types. t-SNE plot of 10,469 primary tumor samples grouped by tumor type via gene expression profiling.

FIGS. 2A, 2B, and 2C show variances and similarities in radiosensitivity and ICI presence among tumors. Violin plots depicting distribution of (2a) RSI values and (2b) ESTIMATE-derived immune scores across 10,469 primary tumor samples, representing 31 tumor types. FIG. 2C shows integration of RSI and the immune score. Blue (radiosensitive) and red (radioresistant) determined by the median RSI value within each tumor type.

FIG. 3 shows non-unimodal distributions in RSI. Hartigan's dip statistic across 31 tumor types to evaluate for unimodal distributions of RSI. Analysis demonstrates that several tumor types do not have a unimodal distribution of RSI values, which suggests biologic heterogeneity.

FIGS. 4A, 4B, 4C, and 4D show distributions of ESTIMATE-derived scores. The ESTIMATE algorithm was used to infer the presence of stromal, immune and malignant cell proportions in 10,469 primary tumor samples in the TCC. Distributions of ESTIMATE-derived stromal and immune scores, as well as tumor purity estimates with associated descriptive statistics.

FIG. 5 shows heterogeneity in stromal cell presence across and within tumor types. Violin plot demonstrating distribution of the ESTIMATE-derived stromal score in 31 tumor types. Demonstrates heterogeneity within and across tumor types for stromal cell presence.

FIG. 6 shows population level relationship between RSI and ESTIMATE-derived immune score. Score plot of principal component analysis of 10,469 primary tumor samples delineated by continuous RSI (left panel) and ESTIMATE-derived immune score (right panel) values based on gene expression.

FIG. 7 shows the correlation between RSI and immune score across individual tumor types. Pearson correlation analysis for RSI and immune score among 31 individual tumor types.

FIGS. 8A, 8B, 8C, 8D, and 8E show IFN signaling pathways influence the immune portraits of intrinsic tumor radiosensitivity. FIG. 8A shows the heatmap of differentially expressed probesets (n=197, representing 146 unique genes) between radiosensitive (RSI^{lo}) and radioresistant (RSI^{hi}) tumors across six or more of the 31 tumor types, which are concordant in direction of expression (up, red; down, blue; no change, gray). See Table 3 for list of probesets. FIG. 8B shows RSI^{lo} versus RSI^{hi} tumors were compared with single sample gene set enrichment analysis (ssGSEA) using the MSigDB hallmark pathway genes related to immune signaling. Heatmap intensity depicts the −log p-value of the comparison test multiplied by the directionality of expression difference. FIG. 8C shows individual genes (n=97) of the MSigDB IFNα hallmark pathway. Tumor types are ordered by the ESTIMATE-derived immune score and heatmap intensity depicts the −log p-value of the comparison test multiplied by the directionality of expression difference of RSI^{lo} versus RSI^{hi} tumors. FIG. 8D shows differentially expressed probesets (146 genes) from FIG. 2a were used as seeds for network generation (see Methods). Genes upregulated (red), downregulated (green) in RSI^{lo} versus RSI^{hi} tumors are depicted. Yellow nodes indicate bridging genes. FIG. 8E shows enrichment of different ICIs between RSI^{lo} and RSI^{hi} tumors. Tumor types are ordered by the ESTIMATE-derived immune scores and normalized CIBERSORT-derived ICI estimates are compared within a given tumor type by dichotomizing at the median RSI value to identify RSI^{lo} and RSI^{hi} tumors. ICIs involved in the innate response (left panel) and ICIs involved in the adaptive response (right panel). Black bars, proportion of tumor types for given ICI enrichment comparison between RSI^{lo} and RSI^{hi} tumors; false-discovery rate<0.05.

FIG. 9 shows various immune-related pathways are upregulated in radiosensitive tumors. Heatmap representing the -log p-values of individual gene expression differences of MSigDB hallmark immune pathways between RSI^{lo} and RSI^{hi} tumors. Pathways include those related to IFNα, IFNγ, allograft rejection, interleukin-6-janus kinase-signal transducer and activator of transcription 3, complement, inflammatory response and coagulation signaling.

FIGS. 10A, 10B, 10C, 10D, 10E, 10F, 10G, 10H, 10I, 10J, 10K, and 10L show integrative in silico modeling defines the tumor immune ecosystem (TIES) and the individual radiation immune sensitivity (iRIS) score. Biologically-defined mechanistic rules guiding tumor cell and ICI interactions in the 3D agent based model (see Methods). A fixed starting malignant cell burden and varying proportions of effector or suppressor ICIs were inputted into the model and simulations resulted in 10a) tumor immune evasion or 10b) immune-mediated tumor elimination. Different populations of cells in the agent-based model are depicted as follows: red (proliferating tumor cells), tan (quiescent tumor cells), green (effector ICIs) and purple (suppressor ICIs). FIG. 10C shows representation of probability of immune-mediated tumor elimination (IMTE) based on individual tumor immune ecosystems (TIES) derived from FIG. 8A and 8B. y-axis: ratio of malignant cell burden/effector ICI (C0/E0); x-axis: ratio of effector/suppressor ICI (E0/S0). Red region, TIES characterized by tumor immune evasion; green region, TIES representing immune-mediated tumor elimination. FIG. 10D shows loading of principal component analysis (PCA) of the CIBERSORT-derived ICI composition across 10,469 tumors stratified as anti-tumor (E; effector) or pro-tumor (S; suppressor). FIG. 10E shows the cellular composition (normalized ICI counts and malignant cell burden estimated by CIBERSORT and ESTIMATE, respectively) for each of the 10,469 tumors was derived and plotted onto the TIES map; all tumors localized in regions with a TIES leading to tumor immune evasion. FIG. 10F shows the visualization of continuous RSI values for each tumor on the TIES map reveals more radiosensitive (RSI^{lo} based on population median) tumors are in closer proximity to TIESs which promote immune-mediated tumor elimination. FIG. 10G shows modeled trajectories of TIES composition evolution of a given pre-treatment TIES following radiation treatment. Each closed circle on the trajectory represents a radiation dose of 2 Gy/day. This demonstrates that radiation causes shifts in the TIES, which result in different trajectories despite being equivalent closest Euclidean distances from the IMTE region (green). FIG. 10H shows the individual radiation immune sensitivity (iRIS) score for each tumor, which describes the specific position in the TIES map with respect to the shortest Euclidean distance from the IMTE region, penalized by the absolute number of suppressor ICIs. A lower iRIS value represents a more radiation responsive TIES. FIG. 10I shows the stratification of tumors by the median RSI and iRIS values distinguishes specific TIES and probability of immune-mediated tumor elimination. FIG. 10J shows scatter plot with correlation of RSI and iRIS across all tumor types. Pearson's r=0.29, P<0.001. Percentages depict proportion of specific TIES in quadrants defined by the median RSI and iRIS values. FIG. 10K shows violin plots depicting distribution of iRIS values, highlighting heterogeneity within and across tumor types. FIG. 10L shows boxplots comparing iRIS distributions between RSI^{lo} and RSI^{hi} tumors (Mann Whitney U test; * P<0.05, **P<0.01, P<0.001). Demonstrates that most radiosensitive tumors are associated with a lower iRIS.

FIG. 11 shows tumor type specific tumor-immune ecosystem (TIES) maps. TIES maps for 31 tumor types showing probability of immune-mediated tumor elimination (IMTE).

FIG. 12 shows the associations between RSI and iRIS and stratification of tumors by integrating both metrics. Tumors were separated into quadrants by RSI^{lo} and RSI^{hi} (population median RSI value), as well as iRIS^{lo} and iRIS^{hi} (population median iRIS value) into quadrants. Pearson correlations across all 31 tumors types between RSI and iRIS.

FIGS. 13A, 13B, 13C, 13D, 13E, 13F, 13G, and 13H show in silico modeling optimizes radiation dose delivery based on the pretreatment TIES in non-small cell lung cancer patients. FIG. 13A shows an independent cohort of 59 non-small cell lung cancer (NSCLC) patients treated with postoperative radiation at various doses (range: 42-70 Gy) were analyzed for locoregional control (LRC), failure (LRF) or overall survival (OS). FIG. 13A shows Kaplan-Meier estimates for OS demonstrate patients with tumors classified as RSI^{lo} vs RSI^{hi} have a trend for improved OS (hazard ratio: 0.58, range: 0.3-1.1; P-value=0.09), whereas iRIS^{lo} vs iRIS^{hi} tumors (hazard ratio: 0.54, range: 0.28-1.0; P-value=0.04) have improved OS. Patient tumors with dual RSP^{lo}/iRIS^{lo} phenotypes have improved OS (hazard ratio: 0.38, range: 0.16-0.92; P-value=0.01) compared to those with RSI^{lo}/iRIS^{hi}, though a RSI^{lo} phenotype with an unfavorable TIES (iRIS^{hi}) or vice versa, cannot compensate for the other (hazard ratio: 1.1, range: 0.4-3.1; P-value=0.84). FIG. 13B shows the cellular composition (ICIs and malignant cell burden) of each patient tumor was plotted onto the TIES map and actual clinical outcomes of LRC (green) and LRF (red) were evaluated with respect to their given TIES. FIG. 13C shows separation of tumors by median RSI and iRIS demonstrates that 83% of RSP^{lo} /iRIS^{lo} tumors achieve LRC compared to 67%, 41% and 33% in RSI^{hi}/iRIS^{lo}, RSI^{hi}/RIS^{hi}, and RSI^{lo}/iRIS^{hi} respectively. FIG. 13D shows (left panel) Representation of a subset (n=15) of analyzed tumors each undergoing in silico modeling according to methods of FIGS. 10a and 10b, with each tumor receiving radiation treatment according to the actual delivered treatment protocol (dose per fraction and total dose). The projected shift in the individual TIES following radiation is depicted by the trajectory; the simulation was continued until complete response (i.e., no tumor cells alive) or treatment failure (i.e. returned to pretreatment tumor burden). The space between each point on a trajectory indicates the change in TIES composition after delivery of a single fraction of radiation. Each individual tumor was grouped according to predicted outcome; LRC (green) or LRF (red). (Right panel) All 59 tumors were plotted onto the TIES map and categorized according to predicted outcome. FIG. 13E shows that each patient tumor underwent in silico modeling with a repeating single 1.8 Gy (circle) or 2 Gy (square) per fraction regimen (based on actual delivered treatment regimens) until complete response was achieved or delivery of at least 60 fractions. After each individual dose the final TIES trajectory was analyzed for response. Plotted coordinates demonstrate the amount of fractions that were necessary to achieve complete response, relative to tumor specific RSI and iRIS continuous values. Notably, several tumors reach at least 60 fractions of radiation without a complete response, whereas some achieve complete response in less than 24 fractions. FIG. 13F shows difference in actual delivered radiation fractions compared to in silico model predicted number of fractions needed to achieve complete response. Negative values imply a decrease in total fractions, which may provide opportunity for de-escalation of treatment, whereas positive values suggest an increased number of fractions may be needed for optimal tumor control. FIG. 13G shows three selected patient tumors (circled tumors in FIG. 13f) with predicted TIES shifts following simulated radiation illustrate three separate scenarios: FIG. 13H shows tumor 1) TIES promotes accelerated tumor eradication and potential for de-escalation of dose, Tumor 2) TIES promotes tumor eradication, but requires more radiation priming, or Tumor 3) TIES is immunosuppressive and only results in tumor kill after several rounds of cytotoxic radiation without assistance from the host immune system. Gray shaded area indicates course of fractionated radiation over a given time.

FIGS. 14A, 14B, 14C, 14D, 14E, 14F, and 14G show the total radiation dose in NSCLC cohort was not informed by RSI or iRIS, but TIES did predict for enhanced tumor control. FIGS. 14A and 14B show RSI and iRIS were not associated with the total radiation dose delivered to the 59 NSCLC patients. FIG. 14C shows that RSI and iRIS were not correlated.

FIG. 14D shows patient tumors that achieved LRC had 14d) higher effector/suppressor ICI and 14e) lower malignant cell/effector ICI ratios than those with LRF. FIG. 14F shows patient tumors that achieved LRC had lower iRIS values compared to those with LRF (P<0.01), where 14G) there was no difference in RSI values between patients with LRC or LRF.

FIG. 15A, 15B, and 15C show cell decision iterative flow diagrams. FIG. 15A shows cancer cell decision making process. FIG. 15B shows immune cells motility process. FIG. 15C shows cell-cell interactions process.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Assessing Radiosensitivity and Treating Cancer

Disclosed herein is a clinical decision support tool that uses individual radiation immune sensitivity (iRIS) as a metric to predict radiation response based on its effect on the tumor-immune ecosystem (TIES). Radiation therapy sensitivity was estimated using a 10 gene radio sensitivity index (RSI) signature, whereby RSI^{lo} refers to tumors that are radiosensitive and RSI^{hi} refers to tumors that are radioresistant.

In one aspect, disclosed herein are methods of detecting the radiosensitivity of a tumor in a subject comprising obtaining a tumor tissue sample, measuring the individual radiation immune sensitivity (iRIS) phenotype of the tumor tissue sample by measuring the pre-radiation distance of tumor-immune ecosystem (TIES) to the immune mediated tumor elimination (IMTE) region, and the number of effector, suppressor and cancer cells; wherein a iRIS value lower than a median iRIS score (such as, for example, an iRIS score of 3.61) is designated as iRIS^{lo} and indicates that a tumor is radiosensitive and an iRIS value above the median iRIS score (i.e., iRIS^{hi}) indicates that a tumor is radioresistant.

It is understood and herein contemplated that the iRIS median score can depend on the TIES. In one aspect, the iRIS median score can be between 3.5 and 3.75, more preferably between 3.55 and 3.7; most preferably between 3.6 and 3.65. For example, the median iRIS score can be 3.5, 3.51, 3.52, 3.53, 3.54, 3.55, 3.56, 3.57, 3.58, 3.59, 3.6, 3.61, 3.62, 3.63, 3.64, 3.65, 3.66, 3.67, 3.68, 3.69, 3.7, 3.71, 3.72, 3.73, 3.74, or 3.75.

Also disclosed herein are methods of detecting the radiosensitivity of a tumor of any preceding aspect, further comprising measuring radio sensitivity index (RSI) of the tumor tissue sample and identifying the cell populations as RSI^{lo} or RSI^{hi} based on the RSI value compared to a median RSI score; wherein a RSI value lower than a median RSI score (such as, for example 0.42) is designated as RSI^{lo} and indicates that a tumor is radiosensitive and an RSI value above the median RSI score (i.e., RSI^{hi}) indicates that a tumor is radioresistant; and wherein a combined iRIS/RSI phenotype of iRIS^{lo}/RSI^{lo} indicates that the tumor is radiosensitive and wherein a combined phenotype of iRIS^{lo}/RSI^{hi}, iRIS^{hi}/RSI^{lo}, or iRIS^{hi}/RSI^{hi} indicates that eh tumor is radioresistant. It is understood and herein contemplated that the RSI median score can depend on the tumor being measured. In one aspect, the RSI median score can be between 0.3 and 0.5, more preferably between 0.35 and 0.45; most preferably between 0.4 and 0.45. For example, the median RSI score can be 0.30, 0.31, 0.32, 0.33, 0.34, 0.35, 0.36, 0.37, 0.38, 00.39, 0.40, 0.41, 0.42, 0.43, 0.44, 0.45, 0.46, 0.47, 0.48, 0.49, or 0.50.

In one aspect, disclosed herein are methods of detecting the radiosensitivity of a tumor of any preceding aspect, further comprising administering fewer fractions of a total radiation dose (such as, for example, less than 20 administered fractions between 1.5 and 2.5 GY for a total administered dose less than 50 GY) to a subject with a tumor that has an iRIS^{lo}/RSI^{lo} phenotype or administering more fractions of a total radiation dose (such as, for example, greater than 20 administered fractions for a total administered dose greater than 30 GY) to a subject with a tumor that has an iRIS^{lo}/RSI^{hi}, iRIS^{hi}/RSI^{lo}, or iRIS^{hi}/RSI^{hi} phenotype.

It is contemplated herein that that the total measured dosage of radiation can be based on the number of fractions administered wherein the total dosage equals the number of fractions multiplied by the dosage per fraction. In one aspect, the dosage per fraction can be between 1.5 and 2.5 GY, more preferably between about 1.8 and 2.0 GY. Thus, a tumor that has an iRIS^{lo}/RSI^{lo} phenotype would receive 20 or less administered fractions (for example, 1, 2, 3, 4, 5, 6, 7, 8, 9, 10, 11, 12, 13, 14, 15, 16, 17, 18, 19, or 20) and preferably less than 1 fractions of a measured fraction of between 1.5 and 2.5 GY, more preferably between about 1.8 and 2.0 GY (for example, 1.5, 1.6, 1.7, 1.8, 1.9, 2.0, 2.1, 2.2, 2.3, 2.4, or 2.5 GY) for a total dosage between 1.8 GY and 50 GY, preferably less than 50GY, more preferably less than 40 GY (for example, between 10.8 GY and 40GY). By contrast, a tumor with a an iRIS^{lo}/RSI^{hi}, iRIS^{hi}/RSI^{lo}, or iRIS^{hi}/RSI^{hi} phenotype would receive greater than 20 administered fractions (for example, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more fractions) of a measured fraction of between 1.5 and 2.5 GY, more preferably between about 1.8 and 2.0 GY (for example, 1.5, 1.6, 1.7, 1.8, 1.9, 2.0, 2.1, 2.2, 2.3, 2.4, or 2.5 GY) for a total dosage between 30 GY and 150 GY, preferably more than 40 GY. Alternatively, dosage can be reduced for each fraction with a tumor with the iRIS^{lo} RSI^{lo} phenotype receiving the same number of fractions as a tumor with the iRIS^{lo}/RSI^{hi}, iRIS^{hi}/RSI^{lo}, or iRIS^{hi}/RSI^{hi} phenotype, but a reduced dosage per fraction relative to the iRIS^{lo}/RSI^{hi}, iRIS^{hi}/RSI^{lo}, or iRIS^{hi}/RSI^{hi} phenotype.

Also disclosed herein are methods of treating, inhibiting, reducing, decreasing, ameliorating, and/or preventing a cancer and/or metastasis in a subject comprising detecting the radiosensitivity as set forth in any preceding aspect and administering to the subject a total dosage of radiation; wherein the total dosage of radiation is administered over several fractions; and wherein the total number of fractions administered is less than 20 when the iRIS/RSI phenotype is iRIS^{lo}/RSI^{lo}; and wherein the total number of fractions administered is greater than 20 when the iRIS/RSI phenotype is iRIS^{lo}/RSI^{hi}, iRIS^{hi}/RSI^{lo}, or iRIS^{hi}/RSI^{hi}. For example, disclosed herein are methods of treating, inhibiting, reducing, decreasing, ameliorating, and/or preventing a cancer and/or metastasis in a subject comprising obtaining a tumor tissue sample from the subject; measuring the individual radiation immune sensitivity (iRIS), wherein a tumor tissue sample from a cancer that has an iRIS score lower than a median iRIS score is designated as iRIS^{lo}; measuring the radio sensitivity index (RSI) of the tumor tissue sample, wherein a RSI value lower than a median RSI score is designated as RSI^{lo}; and administering to the subject a total dosage of radiation; wherein the total dosage of radiation is administered over several fractions; and wherein the total number of fractions administered is less than 20 when the iRIS/RSI phenotype is iRIS^{lo}/RSI^{lo}; and wherein the total number of fractions administered is greater than 20 when the iRIS/RSI phenotype is iRIS^{lo}/RSI^{hi}, iRIS^{hi}/RSI^{lo}, or iRIS^{hi}/RSI^{hi}. In one aspect, the administered dose of radiation can be between 1.5 and 2.5 GY per fraction.

The disclosed compositions can be used to treat any disease where uncontrolled cellular proliferation occurs such as cancers. A non-limiting list of different types of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, pancreatic cancer, or any other type of cancer disclosed in the examples and figures herein.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Harnessing Tumor Immune Ecosystem Dynamics to Personalize Radiotherapy

To better understand the diversity of radiosensitivity in cancer and to identify conserved radiation response modifiers, we previously modeled the radiation response of 48 genetically annotated human cancer cell lines. By integrating the basal transcriptome, TP53 and RAS isoform mutational status, tissue of origin, and clonogenic survival following a radiation dose of 2 Gy (SF2), we identified an interaction network of 474 genes that includes regulators of DNA damage repair (DDR) (e.g. ATM, XRCC6), oxidative stress (e.g. PRDX1, TX1V), as well as ten dominant signaling hubs: AR, JUN, STAT1, PKCB, REM, ABL1, SUMO1, PAK2, HDAC1 and IRF1. From this network we derived the radiosensitivity index (RSI) by training a multi-gene algorithm to predict SF2 in the 48 cells lines. Notably, the radiation response signature is agnostic to cancer type and has been independently validated as a marker of radioresponsiveness across multiple human tumor types, and has recently been proposed as a measurable tumor feature to guide prescription of radiotherapy dose in patients.

The clinical utility of harnessing a patient's own immune system has emerged as a new pillar of tumor therapy, and as such a diverse range of tactics are being investigated to modulate the immune response, including the use of radiotherapy. Indeed, emerging pre-clinical work has shown that irradiated tumors act as adjuvants for the immune system, by facilitating local tumor control and by provoking regression of tumor deposits at distant sites via the abscopal effect. Although synergism has been identified between radiotherapy and the immune system, it is not clear how to optimally integrate radiotherapy in the era of immune-modulating agents. With over three hundred ongoing clinical trials to date combining radiotherapy and immunotherapy, this is a central question in clinical oncology.

Classic radiobiology defines the radiation response as a cellular cytotoxic model based on the 5 Rs (DNA Repair, Repopulation, Reassortment, Reoxygenation and Radiosensitivity). However, the clinical response to radiotherapy occurs in the evolutionary context of complex tumor ecosystems that are comprised of multiple interacting cellular compartments. Using this dynamic system as a framework, we interrogated the relationship between the tumor-immune ecosystem (TIES) and cellular radiosensitivity in solid tumors to inform how one could harness the TIES to optimize delivery of radiotherapy.

To address this need, we initially performed a systematic multi-tier analysis of a cohort of 10,469 transcriptionally profiled, prospectively collected tumor samples representing 31 tumor types. These analyses revealed highly complex relationships between radiophenotype and the TIES that are diverse within and across tumor types. To assess how the interaction of the TIES influences the response of tumors to radiation, we developed an in silico three-dimensional agent-based model that explored the dynamic interplay between tumor cells and the influx/efflux of immune cell populations during tumor development and following radiation. In this model, the TIES is a juxtaposition of two balancing phenotypes, anti-tumor versus pro-tumor, which is based on the proportion of suppressor and effector immune cell infiltrate (ICI) composition of the TIES. Finally, we develop a metric of individual radiation immune sensitivity (iRIS), which explains radiation response based on its effect on the TIES and quantifies the likelihood that radiation can shift the TIES to promote anti-tumor immunity.

### a) Results

### (1) Profiling Defines a Spectrum of Intrinsic Radiation Sensitivity Within and Across Tumor Types

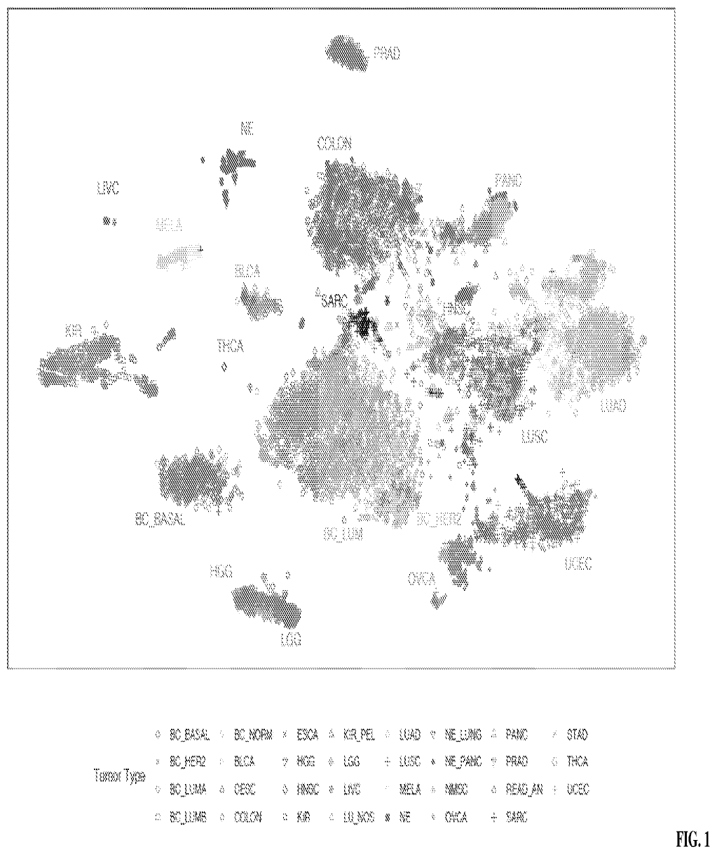

The ‘omics’ era offers many insights into tumor biology and provides a means to classify tumor types based on certain attributes. In this regard, multidimensional reduction analysis of transcription data from 10,469 non-metastatic, primary macrodissected tumors by the t-distributed stochastic neighbor embedding (t-SNE) method demonstrates distinct clustering of tumor types (FIG. 1).

In addition to broadly categorizing tumor types, profiling efforts have provided a basis for targeted therapy (e.g., kinase inhibitors) selection for several malignancies. In contrast, the specific radiation dose and fractionation scheme delivered to a patient's tumor is not currently informed by tumor biology, but is rather guided by clinicopathologic features and decades of empirically derived tolerances of surrounding normal tissues. Further, although substantial experimental and clinical evidence indicate tumors in the same anatomic location and of similar histological subtype can vary widely in their response to radiation, a uniform dosing scheme is generally employed in clinical practice.

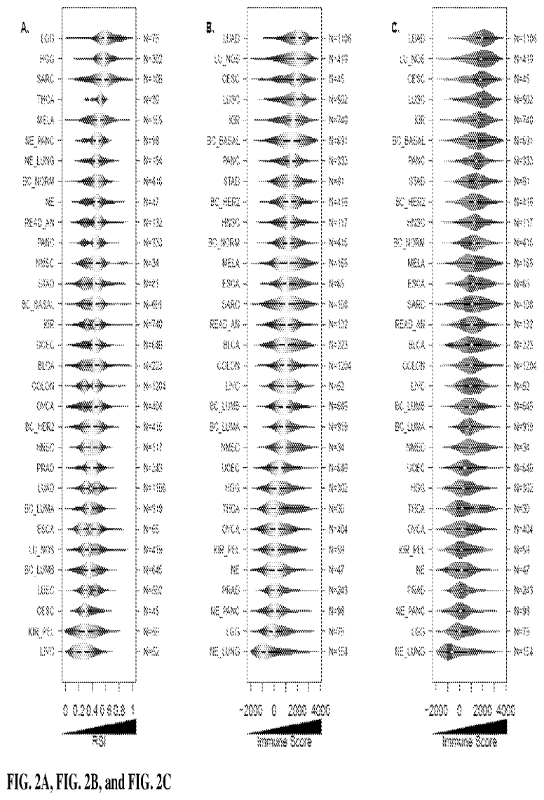

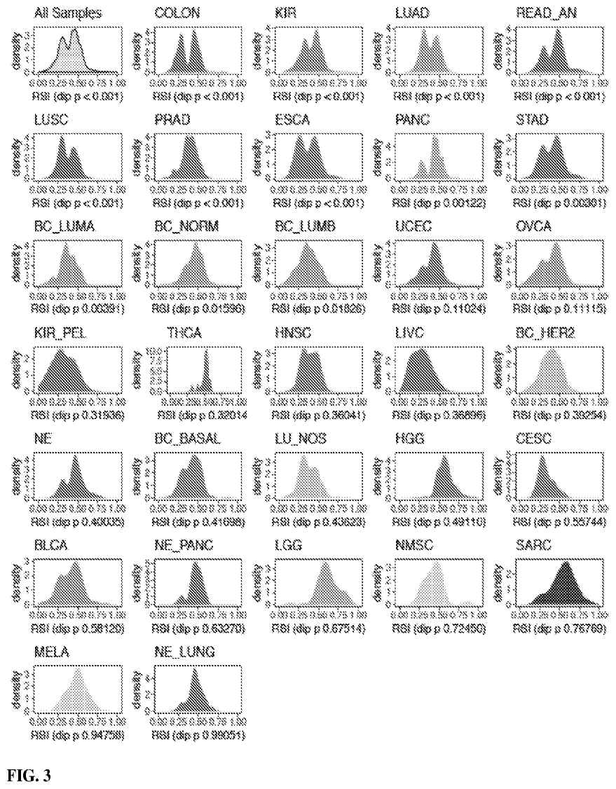

To evaluate the radiosensitivity of human tumors, we estimated intrinsic radiation sensitivity using the radiosensitivity index (RSI). Notably, a spectrum of RSI values within and across the 31 specified tumor types is evident (FIG. 2a). For example, concordant with clinical experience, the most radioresistant (highest median RSI) tumors are gliomas and sarcomas, whereas tumors of the liver, renal pelvis and cervix are the most radiosensitive (lowest median RSI). Stratification of breast cancers into genomically derived subtypes19,20 revealed normal-like breast tumors are the most radioresistant, whereas luminal A and B types are the least (P<0.001). In contrast, no differences in the median RSI are noted between histologic subtypes of lung cancer (P=0.096). Evaluation of interquartile range (IQR) ratios for RSI reveal that renal pelvis, liver, gastrointestinal or genitourinary originating tumors have the greatest dispersion, whereas thyroid and neuroendocrine tumors, and the more radioresistant gliomas, sarcomas and pancreatic tumors are more uniformly distributed (Table 1). Finally as a whole, the RSI distribution among all tumor types is not unimodal based on Hartigan's dip test statistic,21 where several tumor types have non-unimodal distributions in RSI, including kidney, large bowel, rectum-anus, prostate, esophagus, pancreas, stomach, and lung and breast subtypes (FIG. 3)

### (2) Increased ICI Abundance Connotes Radiosensitivity Across Most Tumor Types

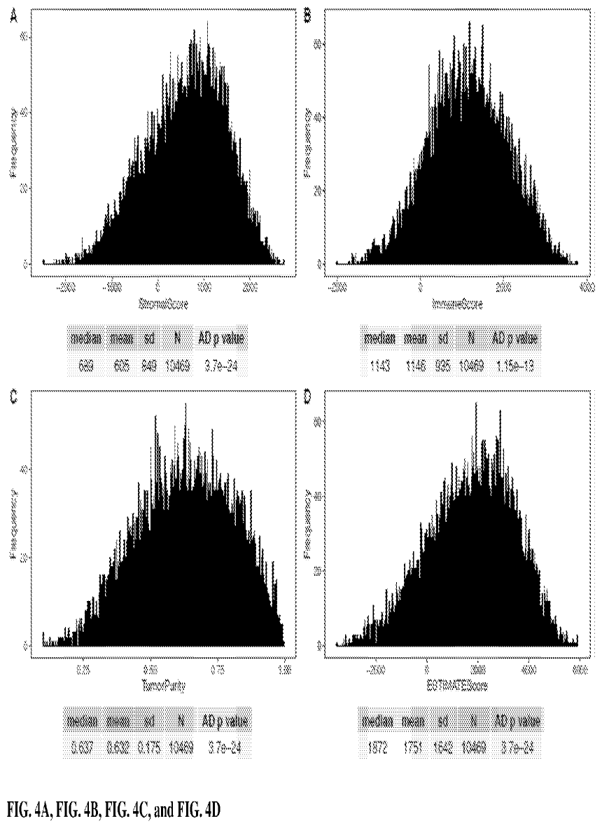

Diverse cell types, including innate and adaptive immune cells and stromal cells, influence the dynamics and radiophenotype of tumors. The ESTIMATE algorithm was employed to infer the fraction of each respective cell type and to approximate tumor purity (i.e., malignant cell burden) within each tumor. The distributions of ESTIMATE-derived stromal, immune and tumor purity scores were visualized for all tumors samples analyzed (FIG. 4).

Similar to the variance observed with the RSI, ESTIMATE-derived approximations of non-tumor cell infiltrates identified a wide range of ICI (FIG. 2b) and stromal (FIG. 5) cells within and across tumor types. Tumor types with clinical responsiveness to immune checkpoint blockade therapies, such as lung, kidney and melanoma, or those largely driven by viral infection (e.g., cervix), have the highest median immune scores, which reflects ICI abundance, especially tumor infiltrating lymphocytes (TIL). Additionally, consistent with prior observations, the breast basal subtype has the highest presence of ICI. Tumors with the lowest median immune scores are gliomas, prostate cancer and neuroendocrine tumors.

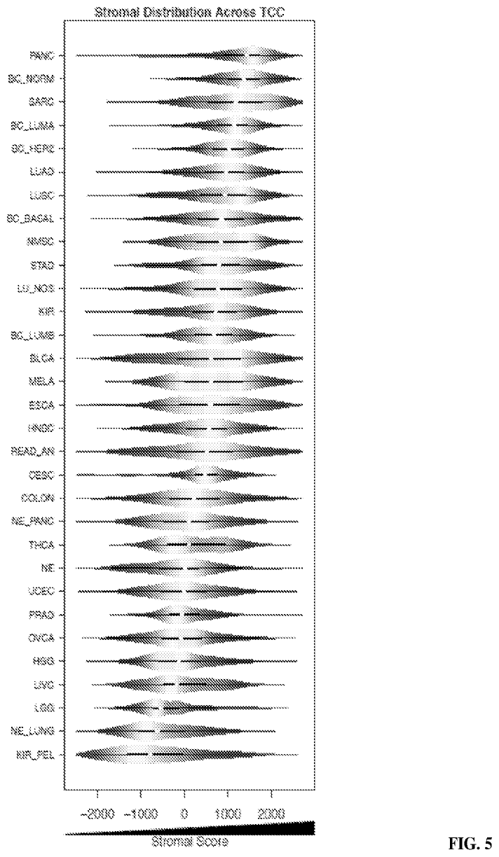

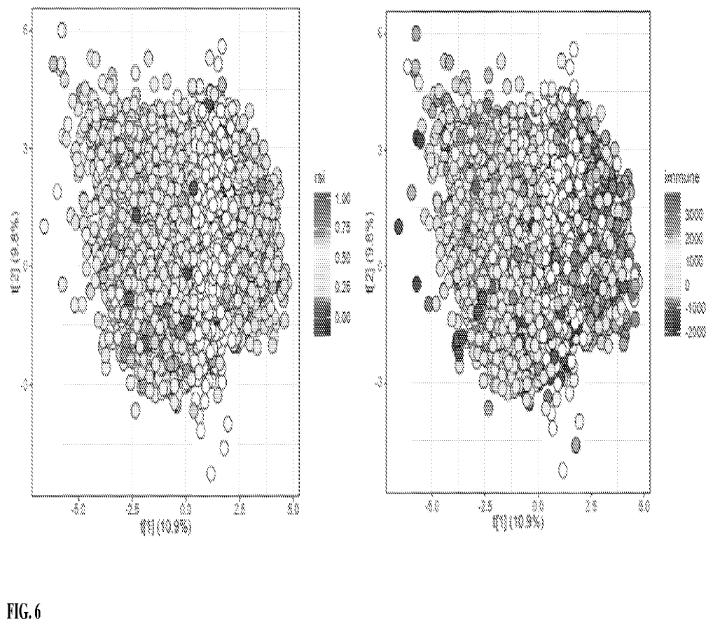

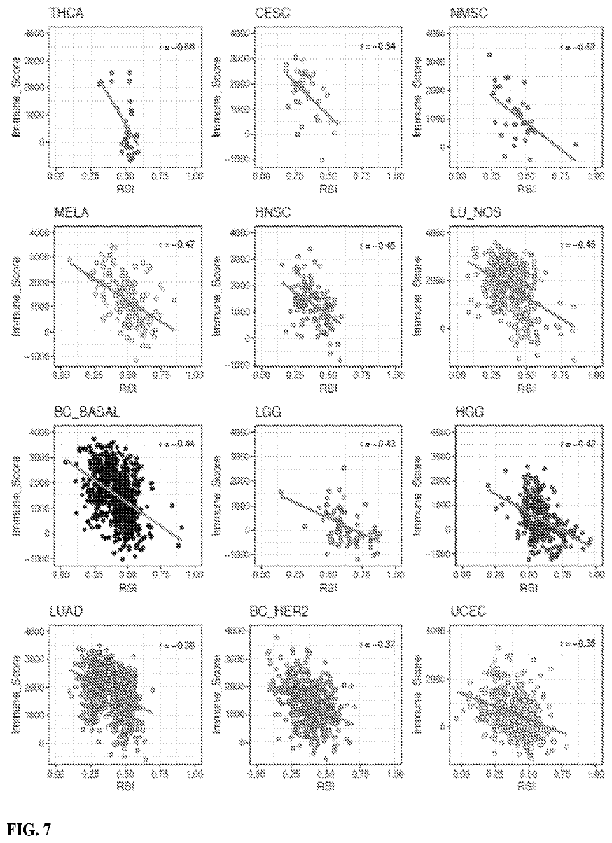

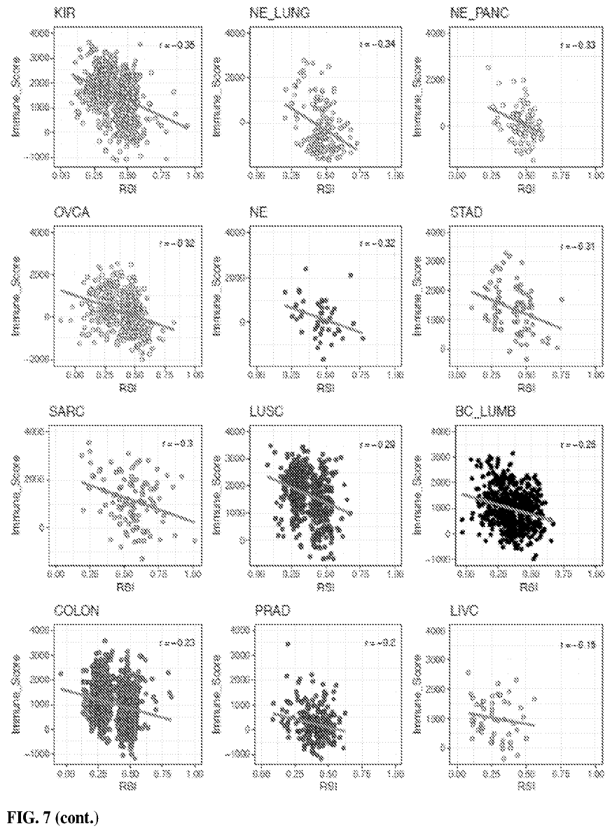

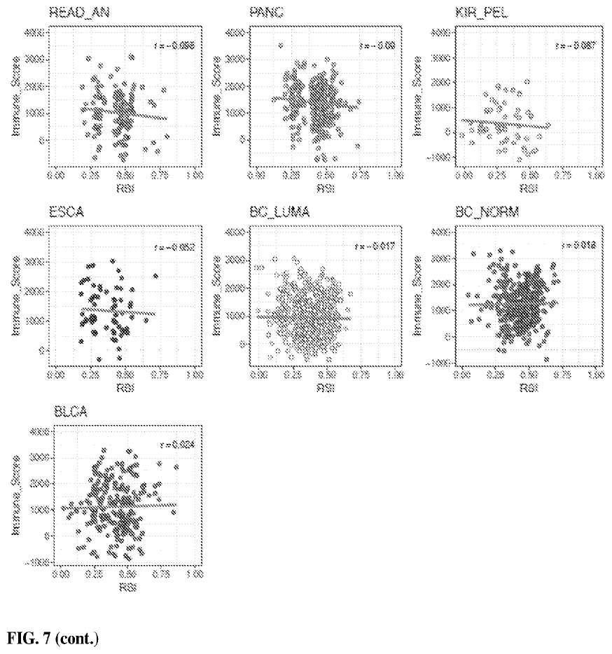

Due to burgeoning data supporting an interplay between radiotherapy and the immune system, as well as data indicating the RSI is partially associated with a 12-chemokine gene signature across several tumor types, we tested if there was a relationship between intrinsic tumor radiosensitivity and the presence of ICI. Among all tumors, there is a weak negative correlation between the RSI and immune score (r=−0.28; P<0.001), yet principal component analysis (PCA) revealed subset aggregation of radiosensitive (i.e., low RSI values) and ICI-rich tumors in the same space (FIG. 6). This indicates radiosensitive tumors having higher ICI. Thus, we evaluated the correlation between the RSI and immune score for each tumor type, which identified moderate negative correlations for thyroid (r=−0.56), cervical (r=−0.54), melanoma (r=−0.47), head and neck (r=−0.45), and basal breast subtype (r=−0.44) tumors; the range of Pearson's r values for all tumor types is −0.56-+0.024 (FIG. 7). Similarly, stromal cell presence (r=0.03) and tumor purity (r=0.13) are weakly related with the RSI. Finally, tumor purity is strongly associated with the immune (r=−0.93) and stromal (r=−0.90) scores.

Next, we classified each tumor sample within a given tumor type as radiosensitive) (RSI^{lo}) radioresistant (RSI^{hi}) based on the median RSI value within a given tumor type. Notably, integration of the RSI and immune score revealed that most radiosensitive tumors are characterized by increased ICI abundance across most tumor types (FIG. 2c). However, there were exceptions, as some tumors classified as radiosensitive had reduced ICI, while others that were radioresistant had high ICI. Overall, the data imply that for most tumor types, ICI presence connotes radiosensitivity.

### (3) IFN Signaling Connotes Intrinsic Tumor Radiosensitivity

To identify the biological discriminators of RSI^{lo} and RSI^{hi} tumors, each tumor type was evaluated for differentially expressed genes (DEGs) (Table 2). Across all tumor types there were 7,184 DEGs between the RSI groups. Interestingly, prostate, neuroendocrine or non-subtyped breast tumors have two or less RSI-influenced DEGs, whereas breast tumor subtypes, except luminal variants, have greater than 150 DEGs between the RSI^{lo} and RSI^{hi} groupings. Furthermore, neuroendocrine tumors stratified as being of pancreas or lung origin unmasked additional DEGs, underscoring the contribution of the microenvironment.

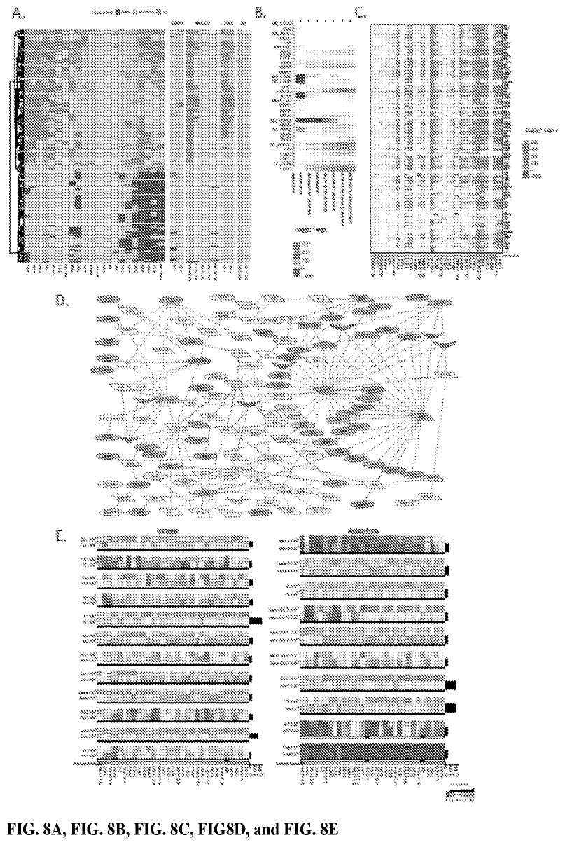

We further investigated whether a conserved RSI-influenced transcriptional program is present in tumors. Notably, these analyses identified 209 unique probesets (155 genes) that are differentially expressed between RSI^{lo} and RSI^{hi} tumors across at least six tumor types, and of these, 146 genes are concordant in their direction of expression (FIG. 8a and Table 3).

Given the relationships between radiosensitivity, immune signaling and the presence of ICI, single sample gene set enrichment analysis (ssGSEA) was performed with the immune-related MSigDB hallmark gene sets. Across several tumor types, ssGSEA revealed that RSI^{lo} versus RSIhi tumors are enriched in pathways regulating type I (α) and II (γ) interferon (IFN) signaling (FIG. 8b). Further, RSI^{lo} tumors having the greatest enrichment of IFNα or IFNγ signaling were mostly manifest in tumor types classified as having the highest immune scores. Evaluation of the IFNα signaling module revealed that numerous genes involved in various aspects of this signaling network are upregulated in RSI^{lo} versus RSI^{hi} tumors (FIG. 8c). Similarly, genes representing six other immune-related pathways were also upregulated in RSI^{lo} tumors across various tumor types (FIG. 9). Pathway topology analyses with the 146 conserved genes identified an expansive interacting network with STAT1, IRF1, and CCL4/MIP-1β as major upregulated nodes in RSI^{lo} tumors (FIG. 8d).

### (4) Tumor Radiosensitivity Correlates With Select ICI Composition

The ESTIMATE-derived immune score provides a generic metric of ICI presence, and does not elucidate the composition or functional state of ICIs. To address the immune repertoire and activation state of tumors, the CIBERSORT deconvolution algorithm was used to infer the presence of 22 distinct immune cell subtypes within each tumor ICI. We found that ICIs involved in the adaptive or innate immune responses were differentially enriched between RSI^{lo} (radiosensitive) and RSI^{hi} (radioresistant) tumors within each tumor type. A common pattern of ICI enrichment was seen in RSI^{lo} versus RSI^{hi} tumors for CD4+ memory T cells (Mem CD4+), CD8+ T cells (CD8+ T), follicular helper T cells (T_{fh}), activated natural killer (NK) cells (NK+) and M1-polarized macrophages (M1) (FIG. 8e; q-value <0.05). However, there is a significant heterogeneity in ICI enrichment both across and within tumor types.

Although relationships between radiosensitivity and ICI presence and composition are apparent, other variables influence both. Indeed, the mutational landscape has been shown to influence tumor immune responses30-34 and previous investigations have identified certain mutations that correlate with radiosensitivity,1,35. To address this relationship we performed targeted sequencing of a subset of tumors (n=2,368) across all types. Notably, mutation frequency (both non-synonymous and synonymous) only weakly correlated with the RSI (r=−0.07; P=0.001), immune score (r=−0.01; P=0.54) and ICI composition (Table 4).

### (5) Dynamic Tumor-Immune Ecosystem Models Define a Novel Individual Radiation Immune Sensitivity Metric

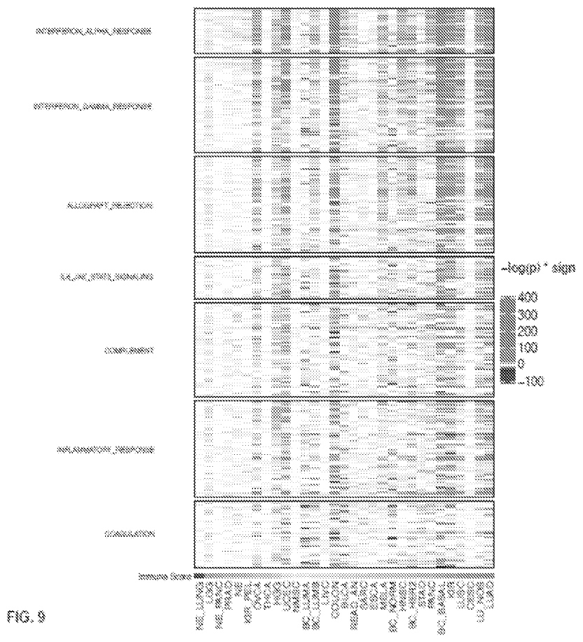

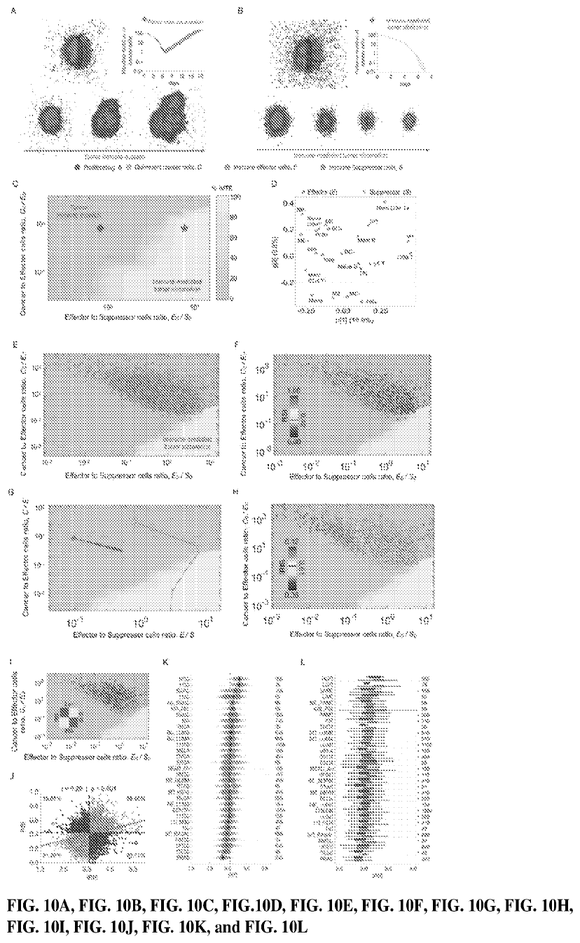

The tumor ecosystem is a dynamic and diverse network of cellular and non-cellular constituents that can either perpetuate or attenuate tumor growth. To model the dynamic interplay between tumor cells and the influx and efflux of ICI components, we generated an in silico 3-dimensional agent-based model guided by biologically defined rules, which incorporates varying proportions of effector and suppressor immune cell populations. The absolute numbers of the effector and suppressor ICI cells along with the cancer cell burden were used to define the tumor-immune ecosystem (TIES, see Methods). Simulation of tumor growth with various TIESs reveals two achievable outcomes, those where tumors evade immune predation (FIG. 10a) and those where tumors are eradicated by the immune system (FIG. 10b). The ratio of cancer cells to effector ICI cells (C0/E0, y-axis) plotted against the ratio of effector ICI cells to suppressor ICI cells (E0/S0, x-axis) spans the “TIES map”, where each pixel represents a unique TIES. Outcome statistics (immune-mediated tumor elimination; IMTE) for ten independent simulations for each initial TIES (C0, E0, S0) identifies TIES compositions that will lead to immune eradication of tumors or, alternatively, that will lead to tumor escape (FIG. 10c).

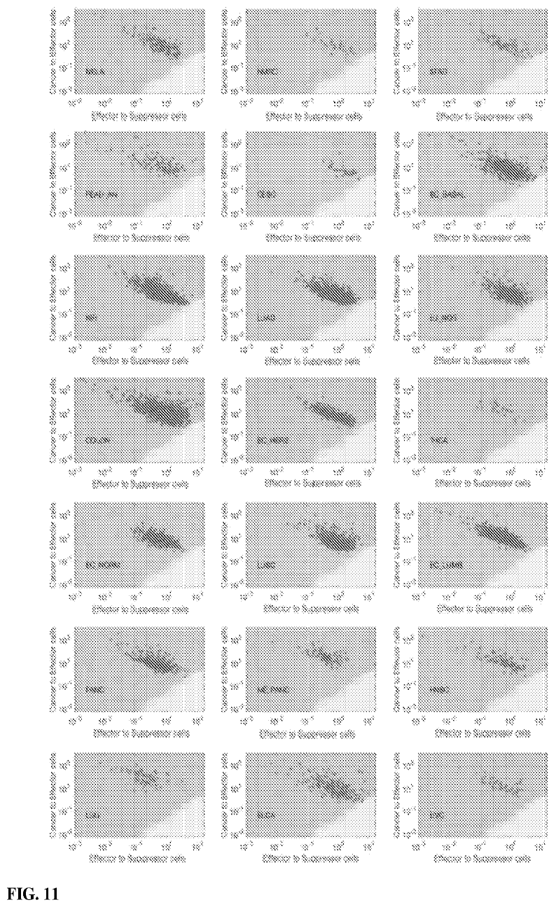

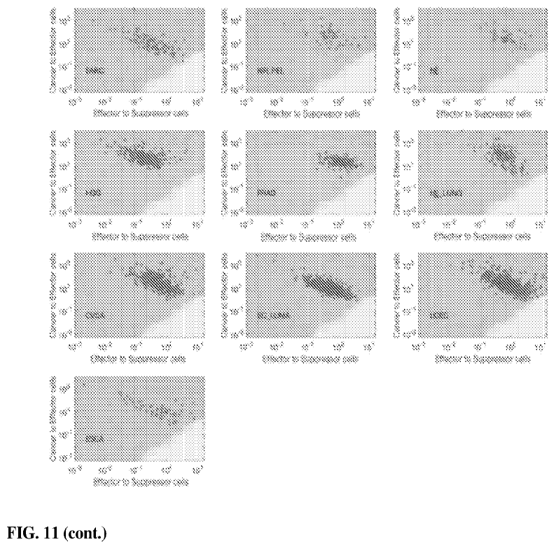

To validate these simulations, the TIES composition for each of the previously analyzed 10,469 tumors was constructed using the ICI counts and malignant cell burden from CIBERSORT-derived assessments and ESTIMATE data, respectively; ICIs were further grouped into effector and suppressor types based on PCA (FIG. 10d). This method identifies that the untreated TIES (C0, E0, S0) of all 10,469 tumors as having a phenotype characterized by immune evasion (FIG. 10e; FIG. 11). Notably, superimposing tumor-specific RSI onto the TIES map indicates that tumors with lower RSI values cluster closer to TIES compositions characterized by immune-mediated tumor elimination (FIG. 10f).

To further evaluate these findings, the effects of radiation on the tumor and the TIES composition were simulated by extending the agent-based model to incorporate radiation induced cytotoxicity of cancer cells and ICIs using experimentally defined radiation sensitivities and ICI responses to radiation (see Methods). This model allows simulation of radiation-induced shifts in TIESs during fractionated radiotherapy for tumors with different pre-treatment TIES compositions. Notably, these simulations revealed that the TIES with more favorable E0/S0 ratios have a higher probability of being shifted to post-radiation TIES compositions that promote immune eradication of residual tumor cells (FIG. 10g). From these findings a new individual radiation immune sensitivity (iRIS) metric was derived with

$$iRIS = \frac{1}{2} min ( {d}_{IMTE} ) + {log}_{10} ( {S}_{0} ) , \\ where \\ \\ {d}_{IMTE} = \sqrt{{( {log}_{10} ( \frac{{E}_{0}}{{S}_{0}} ) - {log}_{10} X )}^{2} + {( {log}_{10} ( \frac{{C}_{0}}{{E}_{0}} ) - {log}_{10} Y )}^{2}}$$

is a function of the distance of pre-radiation TIES composition to the IMTE region in the TIES map (FIG. 10h). Here the X and Y vectors are the coordinates of all TIESs in the region of the IMTE, such that min(d_{IMTE}) denotes the trajectory with the shortest Euclidean distance from the current TIES of immune evasion to one of immune-mediated control.

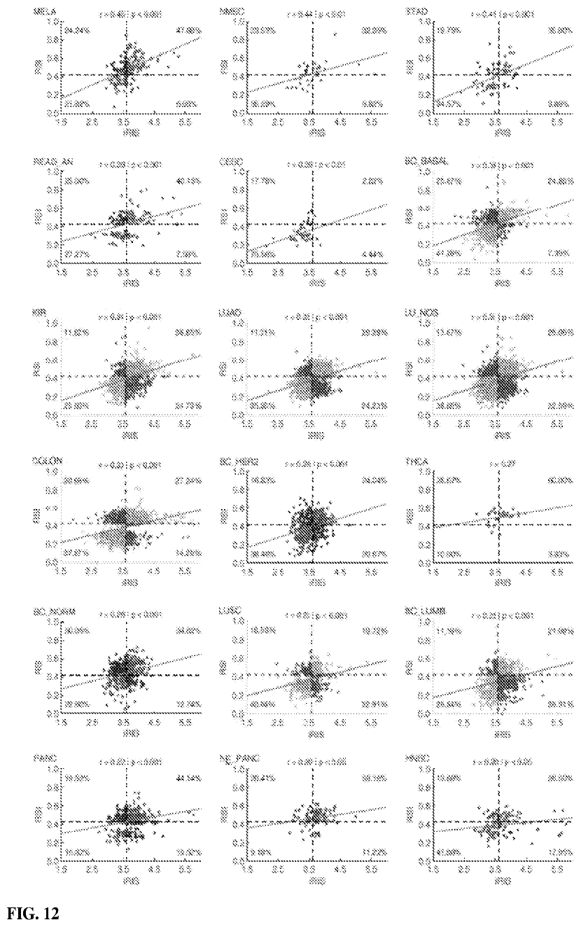

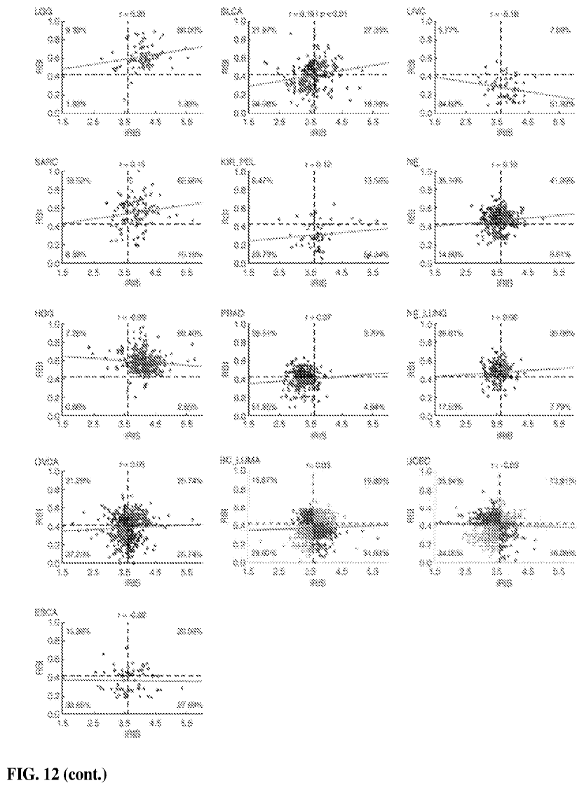

Stratification into RSI^{lo} and RSI^{hi} (median RSI value for all 10,469 tumors), as well as iRIS^{lo} and iRIS^{hi} (median iRIS value) into quadrants, distinguishes TIES composition (FIG. 10i). Specifically, RSI^{lo} tumors have lower iRIS (median 3.52; SD 0.36) than RSI^{hi} (median 3.70; SD 0.42; P<0.001) tumors. Similarly, iRIS^{lo} tumors have lower RSI (median 0.37; SD 0.11) than iRIS^{hi} (median RSI 0.44; SD 0.12; P<0.001) tumors. Accordingly, there is a weak positive correlation between RSI and iRIS across all tumor types (r=0.29; P<0.001, FIG. 10j); the range of Pearson's r values across tumor types is −0.02-+0.45 (FIG. 12). Finally, although iRIS varies across and within different tumor types (FIG. 10k), most RSI^{lo} tumor types have significantly lower iRIS (FIG. 10l).

### (6) RSI and iRIS Mutually Predict Radiation Response in Lung Cancer

To validate these RSI and iRIS findings, an independent clinical cohort of 59 non-small cell lung cancer (NSCLC) patients was examined where patients treated with varying doses of post-operative radiation had overall survival (OS), locoregional control (LRC) and failure (LRF) data.

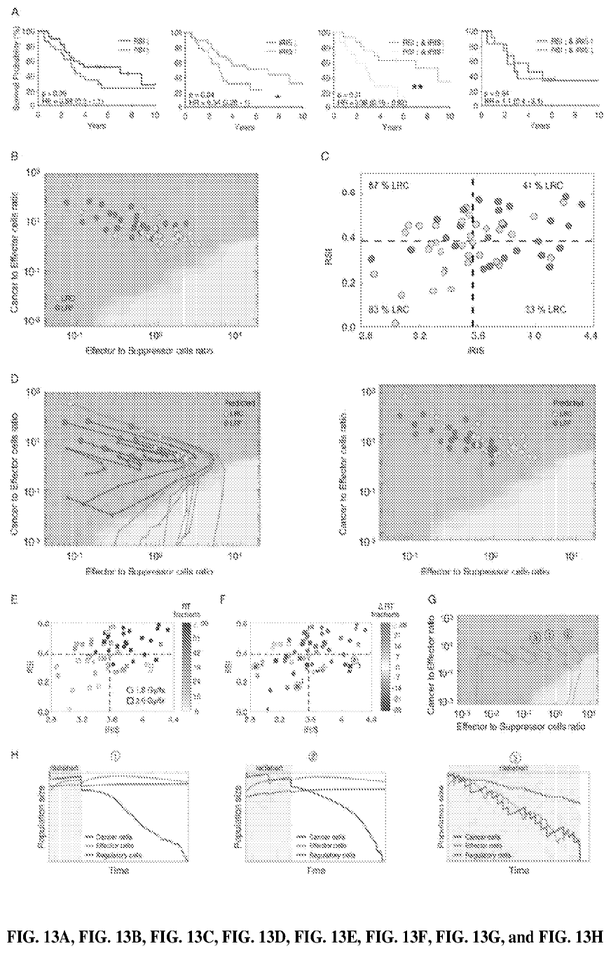

Notably, patients with RSP^{lo} tumors have a trend for improved OS (HR=0.58; P=0.09), and those with iRIS^{lo} versus iRIS^{hi} tumors have improved OS (HR=0.54; P=0.04; FIG. 13a). Further, as predicted, patients with dual RSP^{lo}/iRIS^{lo} tumor phenotypes have superior OS versus patients with RSI^{hi}/RIS^{hi} tumors (HR=0.38 P=0.01; FIG. 13a). Nonetheless, a RSI^{lo} phenotype with an unfavorable TIES (iRIS^{hi}) or vice versa, does not connote improved OS (FIG. 13a); thus, both metrics mutually predict the effects of radiation on immune-mediated tumor eradication.

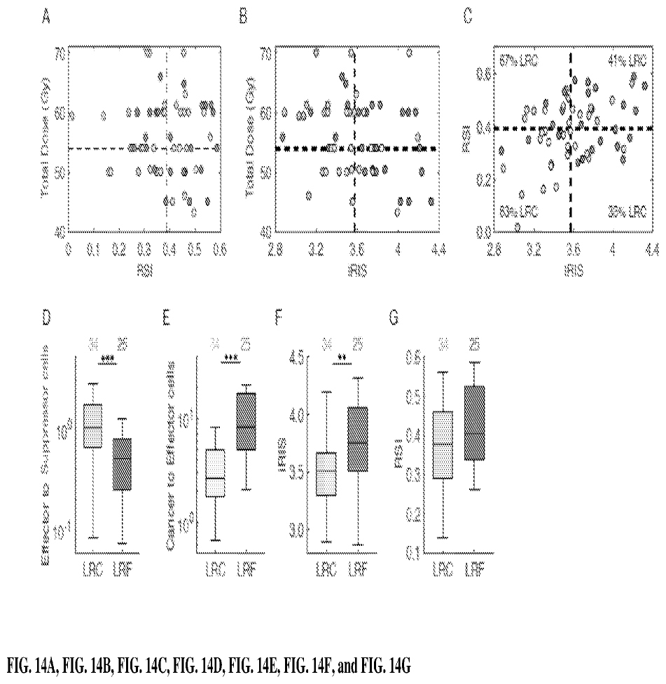

Plotting each patient-specific tumor and their associated outcomes onto the TIES map revealed that most tumors that achieved LRC were in closer proximity to the immune-mediated tumor elimination region (FIG. 13b). Next, to assess the ability to predict patient outcomes, the RSI and iRIS of each tumor was calculated. The total radiation dose received was not informed by RSI or iRIS , nor were RSI or iRIS correlated. Patients that achieved LRC have higher E0/S0 (median 1.02; SD 0.74 vs median 0.51; SD 0.55, P<0.001) and lower C0/E0 ratios (median 2.62; SD 58.25 vs median 8.31; SD 18.16, P<0.001). Achievement of LRC is also associated with lower iRIS (P<0.01), but not RSI (FIG. 14). Patients with favorable radiation sensitivity and TIES phenotypes) (RSI^{lo}/iRIS^{lo} had higher rates of LRC versus those with unfavorable radiation sensitivity and TIES phenotypes (RSI^{hi}/iRIS^{hi}) (83% vs. 41%; P<0.001, FIG. 13c).

### (7) Simulation of Clinically Applied Radiotherapy Dosing Regimens Provides Opportunity to Personalize Radiation Delivery Based on Pre-Treatment TIES Composition

To test if in silico agent-based models can inform personalized treatment, the pre-treatment TIES composition, RSI and iRIS of each NSCLC patient was derived as above. We then integrated these tumor metrics into a simulation to determine the effects of clinically applied individual radiation dosing regimens (total dose applied in 1.8-2 Gy per fractions for a total of 42-70 Gy) to predict radiation-induced shifts in TIES composition. For patients with ‘cold’ TIES (i.e., low E0/S0 ratios) radiation fails to sufficiently perturb the TIES to promote immune-mediated tumor elimination, resulting in LRF. In contrast, in patient tumors with ‘hot’ TIES (i.e. high E0/S0 ratios) predicted to be controlled by radiation, there are radiation-mediated trajectory shifts towards more favorable TIES compositions that support tumor elimination, either by radiation alone or by induced TIES compositions that facilitate immune-mediated eradication (FIG. 13d).

The in silico agent-based models were then used to predict the minimum required number of 1.8 Gy or 2 Gy fractions to eliminate patient-specific tumors. For selected RSI^{lo}/iRIS^{lo}tumors, simulations revealed that as few as 6-10 fractions (10.8-20 Gy total dose) are predicted to be sufficient to provide LRC via robust stimulation of anti-tumor immunity. In contrast, in tumors with an unfavorable RSI^{hi}/iRIS^{hi} TIES compositions, simulations predict up to 60+ fractions (>120 Gy total dose) may be necessary to eradicate every single cancer cell with radiation (FIG. 13e).

Notably, of the 59 analyzed NSCLC patient-specific tumors, 6 (10%) received a radiation regimen within +/−5 fractions of the calculated required dose by the RSI/iRIS-informed in silico model, whereas 30 (51%) are predicted to be candidates for radiation deescalation, and 23 (39%) would require dose escalation by more than 5 fractions (FIG. 13f). Analysis of trajectory shifts in the TIES during radiation for three select patients (FIG. 13g) and the corresponding change in C0, E0, S0 populations over time indicate two different radiation prescription purposes: 1) radiotherapy as an ICI-stimulating agent with opportunity for de-escalated doses (FIG. 13h, left and middle panels) or 2) radiation as a purely cytotoxic agent that has to eradicate every cancer cell without the support of the ICI (FIG. 13h, right panel). Notably in the latter, the persistence of SO populations propagates a microenvironment of radiation-induced, immunosuppressed ICI composition with no observable benefit to radiotherapy.

### b) Discussion

We believe this study represents the largest pan-cancer analysis of primary human tumors characterized as radioresistant or radiosensitive. By employing multi-tier computational analyses, several important tumor features were identified that impact efforts to personalize radiotherapy delivery and to strategically integrate radiotherapy and immunotherapies.

First, RSI, a cancer type agnostic gene signature, allowed characterization of intrinsic tumor radiosensitivity across 31 tumor types, and revealed that RSI predicts clinical experience, where ‘resistant’ (glioma, sarcoma, melanoma) and ‘sensitive’ (cervical, liver) tumors have some of the highest and lowest median RSI values, respectively. Importantly, though differences in radiosensitivity are evident between and within tumor types, the dispersion of RSI values revealed the most sensitive tumors in a given tumor type can overlap with the most resistant tumors of another type and vice versa. Further, characterization of RSI distributions by IQR ratios or dip statistics, establishes that many clinically-defined radioresistant tumors have less dispersion and unimodality, indicating these tumor types lack variation in radioresponsiveness and that tumor control with radiotherapy alone will prove difficult. In contrast, for tumor types with greater dispersion and non-unimodal RSI distributions the data indicates there is an opportunity to personalize radiotherapy to improve the clinical response.

Radiosensitivity is influenced by complex interactions between intrinsic polygenic traits, microenvironment dynamics, utilization of nutrients and diverse cellular composites. Recent studies in 533 tumor cell lines found that intrinsic radiosensitivity is interconnected with DDR and genomic stabilityl, supporting the long held tenet that radiation sensitivity is determined by the fidelity of DDR. However, tumor radiosensitivity is also governed by the other ‘hallmarks of cancer’, and with the success of immunotherapies in the clinic, there is a need to deeply understand relationships between radiation and the host immune response. For this reason, we characterized the presence of ICIs across 31 tumor types and identified a wide spectrum of ICI abundance and composition across and within tumor types. Notably, integration of ICI presence and RSI groupings revealed that across most tumor types, increased ICI presence is associated with a radiosensitive phenotype and that certain ICI compositions are more enriched in radiosensitive versus radioresistant tumors. Similar relationships between radioresponsiveness and select features of the ICI have been identified in breast, prostate, and bladder cancers. Together, the studies indicate the heterogeneity in the repertoire of ICI compositions among tumor types informs strategies that integrate radiation and immunotherapies to improve outcomes.

Recent data have indicated an interesting connection between DDR and antitumor immunity via conserved mechanisms that detect cytosolic nucleic acids to combat foreign pathogens. For example, cyclic GMP-AMP synthase (cGAS)/stimulator of IFN genes (STING) signaling, major regulators of type I IFN production, influence the radiation response. Further, type I IFNs are known to stimulate both the innate and adaptive arms of the immune response and are also essential for radiation efficacy. In accord with these studies, the analyses revealed that RSI^{lo} versus RSI^{hi} tumors are indeed highly enriched in inflammatory signaling, including type I IFNs. Further, although the signaling effectors MB21D1/cGAS and TMEM173/STING failed to meet the statistical criteria for concordant DEGs in a specified number of tumor types between RSI groups, ssGSEA and pathway analysis indicates that radiosensitivity is indeed driven by a STAT1-IRF1-CCL4/MIP-1β network that we submit could be exploited to improve the response to radiation.

The RSI was derived from the NCI-60 cell line panel under uniform culture conditions by modeling the relationship between the basal molecular repertoire and clonogenic survival following a clinically relevant radiation dose. So, how does one explain the dominant ICI signal identified in patient tumors by the RSI, which was derived in cell culture devoid of ICIs? We hypothesized that promiscuous genomic instability and replication stress manifest in these cell lines produces danger associated molecular patterns (DAMPS; e.g., cytosolic DNA) and provokes a chronic IFN-based stress response that was identified in in vitro assays of radiosensitivity. The data are consistent with a model where an evolutionary conserved process of ‘viral mimicry’ that is induced by radiation52 in tumor cells leads to constitutive IFN signaling via shared STAT1-IRF1 transcriptional stress programs despite the heterogeneous origins of tumors.

The findings strongly support the notion that the pretreatment TIES (‘hot’-‘altered’-‘cold’) should be considered when delivering radiation, as the appropriate inflamed state of a tumor following radiotherapy facilitates tumor kill. Though many clinical trials are testing combinations of immunotherapies with radiation, the optimal dose, fractionation, sequencing and timing of these combinations are unknown. To address this need, we developed and then validated an in silico agent-based model that accurately predicts the effects of clinically relevant radiation dosing regimens in tumors harboring varying proportions of interacting effector and suppressor ICIs. Indeed, these simulations predict which TIES s are prone to radiation-induced immune destruction, those that will require priming radiation to shift to a more favorable TIES composition, and those having a resistant TIES where radiation alone will be ineffective. Importantly, these simulations also informed the development of an individual radiation immune sensitivity (iRIS) metric that allows one to more accurately model differences in TIES and personalize radiation dose without compromising efficacy. Specifically, agent-based models informed by each patient-specific pre-treatment ICI composition, RSI and iRIS predicted actual clinical outcomes in NSCLC patients with high accuracy. Critically, this informed model revealed that about half of these lung cancer patients could have de-escalated their radiation dose and still achieved tumor control, and that another 40% required further dose intensification.

The advent of ‘-omics’ analyses has rapidly advanced the understanding of tumor biology and has revealed remarkable heterogeneity of tumor ecosystems and the phenotypes of cells therein. Despite these unequivocal findings, the delivery of radiotherapy, one of the most common therapeutic modalities in oncology, has remained affixed to an imprecise and empiric approach of radiation dose prescription. The era of precision medicine now provides a platform to individualize radiation delivery based on patient-specific tumor attributes coupled with biologically-informed mathematical models. We provide data that two assumed distinct tumor attributes, radiosensitivity and ICI contexture, are linked. Thus, integrating features of the tumor ICI and radiosensitivity provides opportunity to individualize radiation dose delivery and assist with deciding whether treatment should be continued, escalated, de-escalated or changed altogether.

### c) Methods

### (1) Patient Tumor Data Set

Patients were consented to the Total Cancer Care™ (TCC) protocol (IRB-approved, Liberty IRB #12.11.0023). Pathology quality control evaluation of tumors was performed as part of the TCC tissue collection protocol, which includes percent malignant cellularity, necrosis and stromal cell presence . Tumor samples were assayed using the custom Rosetta/Merck HuRSTA_2a520709 Affymetrix gene expression microarray platform (GEO: GPL15048). CEL files were normalized against the median CEL file using IRON, which yields Log2 intensity values per probeset. Principle component analysis (PCA) of all samples revealed that the first principle component was highly correlated to RIN value, indicating a RNA quality difference among samples. A partial least squares (PLS) model was trained upon the fresh frozen samples for which RIN was available and used to re-estimate the RNA quality of all samples. Then the first principle component was removed to correct the signals for RNA quality. 10,469 fresh frozen macrodissected primary tumors were identified for the related analyses.

### (2) Moffitt Non-Small Cell Lung Cancer Cohort

This cohort was obtained from archived tumors that were resected between 2000 and 2010 from patients in the TCC and Moffitt Cancer Center database. All patients provided written informed consent tissue acquisition, molecular profiling and follow-up. We identified 59 tissue samples, which were surgically resected and pathologically confirmed, American Joint Committee on Cancer version 6, stage IIIA-IIIB tumors. Each patient underwent post-operative radiotherapy. Time to recurrence and overall survival was assessed by determination of the treating physician based on clinical source documentation. Gene expression data was obtained from the TCC.

### (3) tSNE

Using the 10,469 samples, we first filtered the 60,607 probesets to those having a standard deviation (across the cohort) >2, yielding 1,399 probesets. The NIPALS algorithm (R/nipals package v0.5) was used to compute the first 50 principal components of the expression data. Rtsne (v0.15) was used with perplexity=100 to generate a two dimensional mapping from these principal components.

### (4) Violin Plots

The violin plots represent the smoothed density with nrd0 bandwidth kernel.

### (5) Estimate

The ESTIMATE algorithm3 was used to evaluate stromal and immune cell fractions from gene expression of the primary tumor (Estimate R package v1.0.13). Normalized gene expression probeset identifiers were mapped to Entrez GeneIDs. As defined in the Estimate package, the data were filtered to only the common genes. A single probeset was then selected per gene by choosing the probeset having the highest median expression across all tumors. This resulting dataset was used in the estimate function to produce stromal and immune scores for each cohort.

### (6) CIBERSORT

The HuRSTA array was reduced to the LM22 signature genes as defined by CIBERSORT4 by choosing a representative probeset that detects the gene and has the highest median expression among matching probesets. The CIBERSORT web tool was accessed on 2017-05-19 to generate signature scores (using quantile normalization).

### (7) Immune Cell Infiltrate (ICI) Normalized Content Abundance

ICI composition proportions from CIBERSORT were extracted in relative mode, where the proportions are relative to the total immune cell fraction of the tumor. To normalize the content across tumors, we scaled the ESTIMATES immune scores such that the lowest immune score was 0 (rather than negative) and analyzed multiple ICI fractions by this adjusted immune score, to yield the Normalized Content Abundance (NCA).

### (8) ICI PCA Plots

Relative ICI composition proportions (CIBERSORT estimates) were used to compute principal components across the entire TCC cohort using prcomp (R3.6.0) with scaling. The loadings and scores were visualized using R packages ggplot2 (3.2.1), ggrepel (0.8.1), and ggpubr (v0.2.3).

### (9) ICI Enrichment

RSI was dichotomized into RSI_HIGH and RSI_LOW by tumor subtype using the median RSI for that type. Each TIL (and ESTIMATE score) was likewise dichotomized using the same approach into ICI_HIGH and ICI_LOW. A Fisher exact test was performed for each comparison of dichotomized RSI and ICI to determine the independence of the variables. The q value package (2.16.0) was used to compute FDR q values to correct for multiple testing (* see additional references). The ICIs were classified as composing adaptive and innate cell types based on current literature and PCA. The enrichment heatmap was generated using ComplexHeatmap (2.0.0). Each cell represents the proportion of samples with ICI_HIGH in either RSI_LO (top row) or RSI_HI (bottom row). The percentage of disease sites with a significant q value (q<0.05) of differences between ICI_HIGH in RSI_LOW vs. RSI_HIGH is noted. * Storey JD. The Positive False Discovery Rate: A Bayesian interpretation and the Qvalue. Annals of Statistics 31, 2013-2035 (2003). * Storey, J D, Taylor J E, and Siegmund D. Strong control, conservative point estimation and simultaneous conservative consistency of False Discovery Rates: A unified approach. J Royal Statistical Society. Series B (Statistical Methodology) 66, 187-205 (2004).

### (10) Calculation of RSI

RSI was calculated for each sample using the within-sample rank coefficients as described. The following 10 probesets, corresponding to the originally defined HG-U133plus2 probesets, were used: merck-NM_007313_s_at (ABL1), merck-NM_000044_a_at (AR), merck-NM_004964_at (HDAC1), merck-NM_002198_at (IRF1), merck-NM_002228_at (JUN), merck-BQ646444_a_at (PAK2, CDK1), merck2-X06318_at (PRKCB), merck2-BC069248_at (RELA), merck-NM_139266_at (STAT1), merck-NM_001005782_s_at (SUMO1).

### (11) ssGSEA

Single-sample GSEA9-11 was used to evaluate the MSigDB Hallmarks Genesets. The GSVA15 package (R package GSVA, v1.32.0) was used to calculate ssGSEA scores. ssGSEA scores were combined by tumor type using the median score.

### (12) RSV^{lo} vs. RSI^{hi}

For probesets that were differentially expressed (DE) between RSP^{lo} and RSI^{hi} within each tumor type, samples were ranked by RSI score and divided into RSI^{lo} and RSI^{hi} groups at the median RSI value. A probeset was determined to be DE within a tumor type if the following criteria were met: (i) probeset must not be annotated as antisense; (ii) one of the two group averages must be >5; (iii) |log2 ratio|≥˜0.585 (1.5-fold); (iv) two tailed t-test≤0.01; and (v) Hellinger distance≥0.25.

### (13) Metacore Pathway

Probesets that were identified as DE in ≥6 tumor types and that had no conflicting direction of change (197 probesets, 146 genes) were used as seeds for MetaCore (Clarivate Analytics) literature network generation. Networks were generated using the Build Network-Shortest Paths method with the following non-default settings: (i) do not show disconnected seed nodes; (ii) do not show shortest path edges only; and (iii) use only Phosphorylation, Dephosphorylation, and Transcription Regulation interaction types of known Activation/Inhibition status. The resulting interaction network was exported and further filtered to contain only nodes and edges consistent with the observed RSP^{lo} vs. RSI^{hi} directions of change, which were then visualized using Cytoscape v3.3.

### (14) DNA Sequencing and Analysis

Sequencing analysis are described. Briefly, coding regions of 1,321 genes were targeted with SureSelect (Agilent Technologies, Santa Clara, Calif.) custom capture and sequenced on GAIIx sequencing instruments (Illumina, Inc., San Diego, Calif.) in paired-end 90 bp configuration. Sequence reads were aligned to the reference human genome with the Burrows-Wheeler Aligner (BWA)18, and duplicate identification, insertion/deletion realignment, quality score recalibration, and variant identification were performed with PICARD and the Genome Analysis ToolKit (GATK)19. Genotypes were determined with GATK UnifiedGenotyper and refined using Variant Quality Score Recalibration. Variants were filtered based on quality (highest sensitivity tranche were excluded) and population frequency (variant observed in 1000 Genomes Project or NHLBI Exome Sequencing Project or at >1% in 238 normal samples from the TCC cohort were excluded). Sequence variants were annotated to determine genic context (ie, non-synonymous, splicing) using ANNOVAR.

### (15) Statistics

Statistical analysis was done using R 3.6.0. The RSI distribution across all tumor types and for each specific tumor type was tested for being unimodal using Hartigan's dip test (R package diptest, v0.75-7) and visualized using ggplot2 (3.2.1) and ggpubr (v0.2.3). Distributions of ESTIMATE scores (immune, stromal, tumor purity, ESTIMATE) were tested for normality using the Anderson-Darling test for normality (R package nortest, v1.0-4) and visualized using ggplot2 (v3.2.1) and ggpubr (v0.2.3). RSI and Immune score were compared using Pearson correlation (r) and visualized using ggpubr (v0.2.3). RSI and all ESTIMATE scores (stromal, ESTIMATE, immune and tumor purity) were compared using GGally (v1.4.0).

### d) Mathematical Modeling Methods

### (1) Agent-Based Model Infrastructure

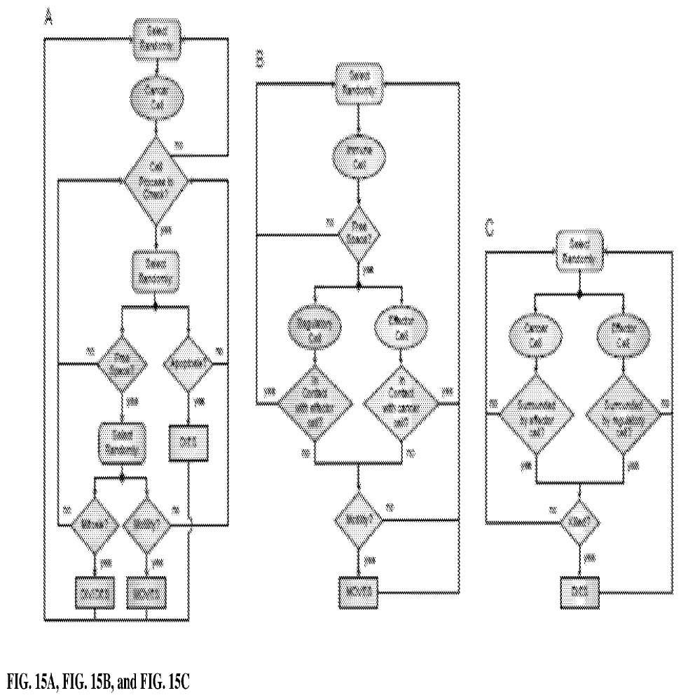

We developed a three-dimensional (3D) multiscale agent-based model that simulates the interactions of cancer cells with anti-tumor immune effector T-cells and immune-inhibitory suppressor cells. Each cell is considered as an individual agent, and their behavior at any time is determined by a stochastic decision making process based on biological-driven mechanistic rules (see refs. 1-4 for similar modeling approaches) Immune cells are simulated on a 3D regular lattice divided into 200×200×200 nodes representing a volume of 65 mm3, with a lattice constant of 20 μm (average cell diameter, ref. 3). Cancer cells occupy an irregular 3D lattice that is initialized by randomly placing one lattice node inside each cube of the regular lattice for immune cells. These interconnected lattices are then used to simulate the infiltration of immune cells into the tumor bulk, thus avoiding contact inhibition between immune and tumor cells. A Moore neighborhood is considered for nodes in the same lattice; i.e., 26 orthogonally and diagonally adjacent lattice sites, where each node has eight neighbor nodes in the opposing lattice given by the nodes surrounding it. Model simulations are initiated with a single cancer cell at the center of the 3D simulation domain. At each time-step ( [symbol] t=1 h), trafficking, motility, cytotoxic function and suppressing activity of immune cells, as well as tumor cell processes are executed through an iterative procedure (FIG. 15). Every time-step the state and dynamics of all cancer and immune cells are updated in random order to avoid order biases. When the tumor reaches a population of 105 cancer cells, the number of cancer cells, immune effectors and suppressor cells define the pre-radiation tumor-immune ecosystem (TIES). The direct dose dependent cytotoxic effect of radiotherapy (RT) on all participating cell types is simulated using the established Linear-Quadratic (LQ) model5-7. Model simulations continue after RT until either the immune system eradicates remaining cancer cells, or recurrent tumors reach the pre-treatment number of 105 tumor cells.

### (2) Cancer Cell Dynamics

Cancer cells proliferate, migrate or undergo programmed cell death (apoptosis) at pre-defined intrinsic probabilities modulated by the local microenvironment (FIG. 15A). Proliferation and migration attempts are only successfully executed for cells residing at a lattice node with at least one vacant adjacent node. During mitosis, the new cancer cell is placed on a randomly selected free neighbor node. Similarly, cells migrate into adjacent vacant lattice nodes with equal probability to simulate random walks and cell diffusion (Brownian motion). The ability of the tumor cell to proliferate and move is temporarily lost due to contact inhibition, and this quiescent state is abandoned as soon as at least one neighbor lattice site becomes free. Cancer cells that undergo programmed cell death with specified probability are removed from the simulation domain instantaneously. For demonstration purpose, we assume commonly used generic cancer cell parameters, including a cell cycle duration of 35 hr (which results in a proliferation probability pp=0.02.31×10-2 per time-step ( [symbol] t=1 h)8), and apoptotic and migration probabilities as pa=4.17×10-3 (approximately every 10 days 9) and pm=4.17×10-1 (about cell widths per day 10).

### (3) Immune Cell Infiltration Into the Tumor Microenvironment

Immune cells arrive in the tumor microenvironment after extravasation from the vasculature. Blood vessels are assumed to be homogeneously distributed throughout the simulation domain, and immune cells enter the simulation at randomly selected free lattice nodes in the domain. At each simulation time-step ti, the number of effector and suppressor cells that are recruited in response to tumor burden is given as:

$${E}_{i} = \{ \begin{matrix}{r}_{E} \cdot {N}_{T} - {N}_{E} if ( {r}_{E} \cdot {N}_{T} ) > {N}_{E} \\ 0 , otherwise\end{matrix} \qquad ( 1 )$$

$${S}_{i} = \{ \begin{matrix}{r}_{S} \cdot {N}_{T} - {N}_{S} if ( {r}_{S} \cdot {N}_{T} ) > {N}_{S} \\ 0 , otherwise\end{matrix} \qquad ( 2 )$$

where NT, NE and NS are the amount of tumor cells (T), effector cells (E) and suppressor cells (S) in the system at time ti. By considering rE=NE/NT and rS=NS/NT, pre-defined ratios of effector-to-tumor cells and effector-to-suppressor cells can be simulated. For patient-specific simulations, the surgical tissue-derived number of cancer, effector and suppressor cells initiate the RT model (C0, E0 S0).

### (4) Immune Cell Motility

Immune cells migrate and infiltrate tumors via chemotaxis along cytokines and chemokine gradients that are secreted by cancer cells or other immune cells. The mean velocity of immune cells in non-lymphoid tissues have been estimated at 4-10 μm min-1, with a peak velocity as high as 25 μm min-1 in the lymph nodes. Here, we set the mean immune cell velocity to 10 μm min-1, which results in a lattice move probability of hr=0.37 per min-1. The waiting time for immune cell movement follows an exponential probability distribution Immune cell motility is updated 60 times (every minute) per simulation time-step ( [symbol] t=1 h). Both effector and suppressor cells move towards the tumor center of mass subject to at least one vacant lattice node in their immediate neighborhood and direction (FIG. 15B) Immune effector and suppressor cells only move while not performing cytotoxic or suppressing functions, i.e., when not in contact with cancer cells or effector cells, respectively.

Immune cells select free neighbor lattice nodes with a certain probability depending on their distance to the center mass of the tumor. The transition probability for the directed movement of an immune cell from a lattice site r to a free neighbor r′ is given by:

$$p ( r \to {r}^{′} ) = \frac{\text{?}}{\sum \text{?}}$$

$$\text{?} \text{indicates text missing or illegible when filed}$$

where V(r) is the set of free neighbor lattice nodes of r, [symbol] H=H(r′)−H(r), and H(j) is the distance of a lattice site j to the tumor center of mass

$$\frac{1}{N} {\sum }_{i = 1}^{N} \overline{{r}_{i}} .$$

Here the vector r! Denotes the spatial coordinates of the lattice node ri occupied by a tumor cell and N is the total amount of tumor cells. A random number is generated to select a free neighbor lattice site for movement, which is determined from the set of probabilities into which the generated number falls in. We fixed η=0.25, which results in a non-deterministic biased random walk.

### (5) Cell-Cell Interactions

Accumulating evidence demonstrates cancer cells secrete a wide array of different chemokines and chemotactic cytokines that recruit pro- and anti-tumor immune cell subsets to the tumor microenvironment. While cytotoxic effector T cells have the potential to kill cancer cells by inducing different forms of cell death, suppressor T cells suppress anti-tumor immunity by inhibiting the cytotoxic responses of effector T-cells. Accordingly, effector T cells can kill cancer cells by direct contact with probability pE=0.03 per time-step, and suppressor T cells can suppress effector-dependent responses by repressing effector T cells when they are in contact at a probability pR=0.01 per time-step (FIG. 15C). Tumor cell killing and effector T cell inactivation probabilities increase proportionally with the number of immune cells of the same types in the immediate cancer cell neighborhood. The target cell is removed from the simulation domain.

### (6) Effect of Radiation on Tumor and Immune Cells

The cytotoxic effect of radiotherapy on cancer cells was simulated by using the standard Linear-Quadratic (LQ) model. The radiation dose D (Gy) dependent surviving fraction (SF) is given by:

SF(D)=e^{ξ(αD+βD}^{²}⁾

where α (Gy-1) and β (Gy-2) are cell type-specific radiosensitivity parameters. Growth arrested cells are approximately three-times more resistant to radiation than normoxic cycling cells19; we set ξ=1 and ξ=⅓ to respectively scale the radiosensitivity of proliferative and quiescent tumor cells.

As in silico tumors are about six orders of magnitude smaller than clinically apparent tumors, simulating seven weeks of radiation will overestimate tumor control. With generic cancer radiosensitivity of α=0.3 Gy-1 and β=0.03 Gy-1, we have set SF (2 Gy×30)≅4e-10. With SF (2 Gy×10)≅7e-4, each RT dose in silico is estimated to equate three actual radiotherapy doses. For RT simulations for an individual NSCLC patient k, we set SFk (2 Gy)=RSIk.

Immunosuppressive cells are intrinsically more resistant to radiation than cytotoxic effector T cells 20,21, in line with estimates of radiation-induced lymphocyte death after increasing radiation doses in vitro 22. From these dose-response curves we derive SFS(1.8Gy)=0.81 and SFS(2.0Gy)=0.79 for suppressor immune cells, and SFE(1.8Gy)=0.63 and SFE(2.0Gy)=0.61 for immune effector cells.

### (7) Radiation-Induced Recruitment of Immune Cells

Radiation induces immunogenic cell death, and this releases immune-stimulating signals and enhances T cell infiltration into the TME. Radiation-induced tumor cell death occurs during the first hours after irradiation 26 and is marked by increased infiltration and recruitment of T lymphocytes 27. Radiation-induced effector and suppressor cells recruited at time ti is simulated by:

Eᵢ=Σⱼ₌₁^{N}^{_{RT}}K_{T}_{ⱼ}·(δ_{E}e^{-γ(tj-tj)}).

Sᵢ=Σⱼ₌₁^{N}^{_{RT}}K_{T}_{ⱼ}·(δ_{S}e^{-γ(tj-tj)}).

where NRT is the number of radiation fractions, and KTj is the amount of cancer cells eradicated by radiation at fraction j delivered at time tj. The parameters δE and δS describe radiation-induced recruitment rates of effector and suppressor cells, and γ is the decay of radiation-induced immune stimulation. Parameter values δE=0.05 h−1, δR=0.01 h−1 and γ=0.05 were used for all model simulations.

### (8) Individual Radiation Immune Sensitivity (iRIS) Score

The TIES composition prior to therapy combines cancer-to-effector cell and effector-to-suppressor cell ratios with the absolute number of suppressor cells. Effector (anti-tumor) ICI was represented by CD8 T, activated CD4 memory T, activated NK, and M1-polarized macrophage cells. Suppressor (pro-tumor) ICI was represented by regulatory T, M2-polarized macrophage and neutrophil cells. These ICI type were chosen in line with previous literature and clean signal on PCA. Including all the ICI populations following PCA stratification yields comparable results, but lower significance due to ambitious stratification. To correlate TIES composition with radiation responses the individual radiation immune sensitivity (iRIS) score is defined as:

$$iRIS = \text{?} min ( {d}_{IMTE} ) + {log}_{10} ( {S}_{0} )$$

$$where$$

$${d}_{IMTE =} \sqrt{{( {log}_{10} ( \text{?} ) - {log}_{10} X )}^{2} + {( {log}_{10} ( \text{?} ) - {log}_{10} Y )}^{2}}$$

$$\text{?} \text{indicates text missing or illegible when filed}$$

is the distance of pre-radiation TIES to the immune mediated tumor elimination (IMTE) region, and E0, S0 and C0 are the number of effector, suppressor and cancer cells, respectively. The X and Y vectors are the coordinates of all simulated points in the IMTE region in the immune contexture matrix.

### D. REFERENCES

Abaan, O. D. et al. The exomes of the NCI-60 panel: a genomic resource for cancer biology and systems pharmacology. Cancer Res 73, 4372-4382, doi:10.1158/0008-5472.CAN-12-3342 (2013).

Abazeed, M. E. et al. Integrative radiogenomic profiling of squamous cell lung cancer. Cancer Res 73, 6289-6298, doi:10.1158/0008-5472.CAN-13-1616 (2013).

Ahmed, K. A. et al. Radiosensitivity Differences Between Liver Metastases Based on Primary Histology Suggest Implications for Clinical Outcomes After Stereotactic Body Radiation Therapy. Int J Radiat Oncol Biol Phys 95, 1399-1404, doi:10.1016/j.ijrobp.2016.03.050 (2016).

Ahmed, K. A. et al. Radiosensitivity of Lung Metastases by Primary Histology and Implications for Stereotactic Body Radiation Therapy Using the Genomically Adjusted Radiation Dose. J Thorac Oncol 13, 1121-1127, doi:10.1016/j.jtho.2018.04.027 (2018).

Ahmed, K. A. et al. The radiosensitivity index predicts for overall survival in glioblastoma. Oncotarget 6, 34414-34422, doi:10.18632/oncotarget.5437 (2015).

Ahmed, K. A. et al. The radiosensitivity of brain metastases based upon primary histology utilizing a multigene index of tumor radiosensitivity. Neuro Oncol 19, 1145-1146, doi:10.1093/neuonc/nox043 (2017).

Alfonso J, Schaadt N, Schonmeyer R, Brieu N, Forestier G, Wemmert C, Feuerhake F, and Hatzikirou H., In-silico insights on the prognostic potential of immune cell infiltration patterns in the breast lobular epithelium. Scientific Reports 6: 33322 (2016).

Alfonso J C L, Jagiella N, Nunez L, Herrero M A, and Drasdo D. Estimating dose painting effects in radiotherapy: a mathematical model. PloS One 9: e89380 (2014).

Anderson A, and Rejniak K. Single-cell-based models in biology and medicine. Springer Science & Business Media (2007).

Aran, D., Sirota, M. & Butte, A. J. Systematic pan-cancer analysis of tumour purity. Nat Commun 6, 8971, doi:10.1038/ncomms9971 (2015).

Bakhoum, S. F. et al. Chromosomal instability drives metastasis through a cytosolic DNA response. Nature 553, 467-472, doi:10.1038/nature 25432 (2018).

Barbie, D. A. et al. Systematic RNA interference reveals that oncogenic KRAS-driven cancers require TBK1. Nature 462, 108-112, doi:10.1038/nature 08460 (2009).

Barker H E, Paget J T, Khan A A, and Harrington K J. The tumour microenvironment after radiotherapy: mechanisms of resistance and recurrence. Nature Reviews Cancer 15: 409-425 (2015).

Bellone M, and Calcinotto A. Ways to enhance lymphocyte trafficking into tumors and fitness of tumor infiltrating lymphocytes. Frontiers in Oncol 3: 231 (2013).

Binnewies M, Roberts E W, Kersten K, et al. Understanding the tumor immune microenvironment (TIME) for effective therapy. Nat. Med. 24: 541-550 (2018).

Boss, M. K., Bristow, R. & Dewhirst, M. W. Linking the history of radiation biology to the hallmarks of cancer. Radiat Res 181, 561-577, doi:10.1667/RR13675.1 (2014).

Burnette, B. C. et al. The efficacy of radiotherapy relies upon induction of type I interferon-dependent innate and adaptive immunity. Cancer Res 71, 2488-2496, doi:10.1158/0008-5472.CAN-10-2820 (2011).

Campesato, L. F. et al. Comprehensive cancer-gene panels can be used to estimate mutational load and predict clinical benefit to PD-1 blockade in clinical practice. Oncotarget 6, 34221-34227, doi:10.18632/oncotarget.5950 (2015).

Chaplain M A J, Graziano L, and Preziosi L. Mathematical modelling of the loss of tissue compression responsiveness and its role in solid tumour development. Mathematical Medicine and Biology 23.3: 197-229 (2006).

Chow M T and Luster A D. Chemokines in cancer. Cancer Immunol. Res 2: 1125-1131 (2014).

Citrin, D. E. & Mitchell, J. B. Mechanisms of Normal Tissue Injury From Irradiation. Semin Radiat Oncol 27, 316-324, doi:10.1016/j.semradonc.2017.04.001 (2017).

Cui, Y., Li, B., Pollom, E. L., Horst, K. & Li, R. Integrating Radiosensitivity and Immune Gene Signatures for Predicting Benefit of Radiotherapy in Breast Cancer. Clin Cancer Res, doi:10.1158/1078-0432.CCR-18-0825 (2018).

Deng, L. et al. STING-Dependent Cytosolic DNA Sensing Promotes Radiation-Induced Type I Interferon-Dependent Antitumor Immunity in Immunogenic Tumors. Immunity 41, 843-852, doi:10.1016/j.immuni.2014.10.019 (2014).

Denham, J. W. & Hauer-Jensen, M. The radiotherapeutic injury—a complex ‘wound’. Radiother Oncol 63, 129-145, doi:10.1016/50167-8140(02)00060-9 (2002).

DePristo, M. A. et al. A framework for variation discovery and genotyping using next generation DNA sequencing data. Nat Genet 43, 491-498, doi:10.1038/ng.806 (2011).

Doroshow, J. H. & Kummar, S. Translational research in oncology—10 years of progress and future prospects. Nat Rev Clin Oncol 11, 649-662, doi:10.1038/nrclinonc.2014.158 (2014).

Efstathiou, J. A. et al. Impact of Immune and Stromal Infiltration on Outcomes Following Bladder-Sparing Trimodality Therapy for Muscle-Invasive Bladder Cancer. Eur Urol 76, 59-68, doi:10.1016/j.eururo.2019.01.011 (2019).

Enderling H, Park D, Hlatky L, and Hahnfeldt P. The importance of spatial distribution of stemness and proliferation state in determining tumor radioresponse. Mathematical Modelling of Natural Phenomena 4: 117-133 (2009).

Eschrich, S. A. et al. A gene expression model of intrinsic tumor radiosensitivity: prediction of response and prognosis after chemoradiation. Int J Radiat Oncol Biol Phys 75, 489-496, doi:10.1016/j.ijrobp.2009.06.014 (2009).

Eschrich, S. A. et al. Validation of a radiosensitivity molecular signature in breast cancer. Clin Cancer Res 18, 5134-5143, doi:10.1158/1078-0432.CCR-12-0891 (2012).

Eschrich, S. et al. Systems biology modeling of the radiation sensitivity network: a biomarker discovery platform. Int J Radiat Oncol Biol Phys 75, 497-505, doi:10.1016/j.ijrobp.2009.05.056 (2009).

Fenstermacher, D. A., Wenham, R. M., Rollison, D. E. & Dalton, W. S. Implementing personalized medicine in a cancer center. Cancer J 17, 528-536, doi:10.1097/PPO.0b013e318238216e (2011).

Formenti S C and Demaria S. Combining radiotherapy and cancer immunotherapy: a paradigm shift. J Natl Cancer Inst 105:256-265 (2013).

Formenti S C, Demaria S: Radiotherapy effects on anti-tumor immunity: Implications for cancer treatment. Front Oncol 3:128 (2013).

Fowler J F. The linear-quadratic formula and progress in fractionated radiotherapy. Br J Radiol 62: 679-694 (1989).

Galon, J. & Bruni, D. Approaches to treat immune hot, altered and cold tumours with combination immunotherapies. Nat Rev Drug Discov 18, 197-218, doi:10.1038/s41573-018-0007-y (2019).

Gentles, A. J. et al. The prognostic landscape of genes and infiltrating immune cells across human cancers. Nat Med 21, 938-945, doi:10.1038/nm.3909 (2015).

Gooden M J, de Bock G H, Leffers N. Daemen T and Nijman H W. The prognostic: influence of tumour-infiltrating lymphocytes in cancer: a systematic review with meta-analysis. British J. Cancer 105: 93-103 (2011).

Gu, Z., Eils, R. & Schlesner, M. Complex heatmaps reveal patterns and correlations in multidimensional genomic data. Bioinformatics 32, 2847-2849, doi:10.1093/bioinformatics/btw313 (2016).

Hanzelmann, S., Castelo, R. & Guinney, J. GSVA: gene set variation analysis for microarray and RNA-seq data. BMC Bioinformatics 14, 7, doi:10.1186/1471-2105-14-7 (2013).

Harrington, K., Jankowska, P. & Hingorani, M. Molecular biology for the radiation oncologist: the 5Rs of radiobiology meet the hallmarks of cancer. Clin Oncol (R Coll Radiol) 19, 561-571, doi:10.1016/j.clon.2007.04.009 (2007).

Hartigan, J. & Hartigan, P. The Dip Test of Unimodality. The Annals of Statistics 13, 70-84 (1985).

Hellmann, M., Rizvi, N., Wolchok, J. D. & Chan, T. A. Genomic profile, smoking, and response to anti-PD-1 therapy in non-small cell lung carcinoma. Mol Cell Oncol 3, e1048929, doi:10.1080/23723556.2015.1048929 (2016).

Herrera F G, Bourhis J, and Coukos G. Radiotherapy combination opportunities leveraging immunity for the next oncology practice. CA Cancer J Clin 67: 65— 85 (2017).

Heuvers, M E, Aerts J G, Cornelissen R, Groen H, Hoogsteden H C, Hegmans J P. Patient-tailored modulation of the immune system may revolutionize future lung cancer treatment. BMC Cancer 12: 580 (2012).

Junttila M R and de Sauvage F J. Influence of tumour micro-environment heterogeneity on therapeutic response. Nature 501: 346-354 (2013).

Kachikwu E L, Iwamoto K S, Liao Y-P, DeMarco J J, Agazaryan N, Economou J S, McBride W H and Schaue D. Radiation enhances regulatory T cell representation. Int J. of Radiation Oncol Biol Physics 81: 1128-1135 (2011).

Kerns, S. L. et al. Radiation biology and oncology in the genomic era. Br J Radiol 91, 20170949, doi:10.1259/bjr.20170949 (2018).

Krug, K. et al. A Curated Resource for Phosphosite-specific Signature Analysis. Mol Cell Proteomics 18, 576-593, doi:10.1074/mcp.TIR118.000943 (2019).

Lee S P, Leu M Y, Smathers J B, et al: Biologically effective dose distribution based on the linear quadratic model and its clinical relevance. Int J Radiat Oncol Biol Phys 33:375-389 (1995).

Li, H. & Durbin, R. Fast and accurate short read alignment with Burrows-Wheeler transform. Bioinformatics 25, 1754-1760, doi:10.1093/bioinformatics/btp324 (2009).

Li, T. & Chen, Z. J. The cGAS-cGAMP-STING pathway connects DNA damage to inflammation, senescence, and cancer. J Exp Med 215, 1287-1299, doi:10.1084/jem.20180139 (2018).

Liberzon, A. et al. Molecular signatures database (MSigDB) 3.0. Bioinformatics 27, 1739-1740, doi:10.1093/bioinformatics/btr260 (2011).

Liberzon, A. et al. The Molecular Signatures Database (MSigDB) hallmark gene set collection. Cell Syst 1, 417-425, doi:10.1016/j.cels.2015.12.004 (2015).

Lim, J. Y., Gerber, S. A., Murphy, S. P. & Lord, E. M. Type I interferons induced by radiation therapy mediate recruitment and effector function of CD8(+) T cells. Cancer Immunol Immunother 63, 259-271, doi:10.1007/s00262-013-1506-7 (2014).

Liu, H. et al. Tumor-derived IFN triggers chronic pathway agonism and sensitivity to ADAR loss. Nat Med 25, 95-102, doi:10.1038/s41591-018-0302-5 (2019).

Lugade A A, Moran J P, Gerber S A, Rose R C, Frelinger J G, and Lord E M. Local radiation therapy of b16 melanoma tumors increases the generation of tumor antigen-specific effector cells that traffic to the tumor. J. Immunol 174: 7516-7523 (2005).

Masopust D and Schenkel J M. The integration of T cell migration, differentiation and function. Nature Reviews Immunol 13: 309 (2013).

McGee, H. M. et al. Targeting the Tumor Microenvironment in Radiation Oncology: Proceedings from the 2018 ASTRO-AACR Research Workshop. Clin Cancer Res, doi:10.1158/1078-0432.CCR-18-3781 (2019).

Miller M J, Wei S H, Parker I, and Cahalan M D. Two-photon imaging of lymphocyte motility and antigen response in intact lymph node. Science 296: 1869-1873 (2002).

Moding, E. J., Kastan, M. B. & Kirsch, D. G. Strategies for optimizing the response of cancer and normal tissues to radiation. Nat Rev Drug Discov 12, 526-542, doi:10.1038/nrd4003 (2013).

Morton C I, Hlatky L, Hahnfeldt P, and Enderling H. Non-stem cancer cell kinetics modulate solid tumor progression. Theoretical Biol and Med Modelling 8: 48 (2011).

Mueller S N. Effector T-cell responses in non-lymphoid tissues: insights from in vivo imaging. Immunol. and Cell Biol. 91: 290-296 (2013).

Newman, A. M. et al. Robust enumeration of cell subsets from tissue expression profiles. Nat Methods 12, 453-457, doi:10.1038/nmeth.3337 (2015).

Ng, K. W., Marshall, E. A., Bell, J. C. & Lam, W. L. cGAS-STING and Cancer: Dichotomous Roles in Tumor Immunity and Development. Trends Immunol 39, 44-54, doi:10.1016/j.it.2017.07.013 (2018).

Ngwa, W. et al. Using immunotherapy to boost the abscopal effect. Nat Rev Cancer 18, 313-322, doi:10.1038/nrc.2018.6 (2018).

Parker, J. S. et al. Supervised risk predictor of breast cancer based on intrinsic subtypes. J Clin Oncol 27, 1160-1167, doi:10.1200/JCO.2008.18.1370 (2009).

Reinhold, W. C. et al. Identification of a predominant co-regulation among kinetochore genes, prospective regulatory elements, and association with genomic instability. PLoSOne 6, e25991, doi:10.1371/journal.pone.0025991 (2011).

Ribas, A. & Wolchok, J. D. Cancer immunotherapy using checkpoint blockade. Science 359, 1350-1355, doi:10.1126/science.aar4060 (2018).

Rizvi, N. A. et al. Cancer immunology. Mutational landscape determines sensitivity to PD-1 blockade in non-small cell lung cancer. Science 348, 124-128, doi:10.1126/science.aaa1348 (2015).

Rockwell S, Dobrucki I T, Kim EY , et al. Hypoxia and radiation therapy: Past history, ongoing research, and future promise. Curr Mol Med 9:442-458 (2009).

Roszik, J. et al. Novel algorithmic approach predicts tumor mutation load and correlates with immunotherapy clinical outcomes using a defined gene mutation set. BMC Med 14, 168, doi:10.1186/s12916-016-0705-4 (2016).

Sachs R K, Hahnfeld P, and Brenner D J. The link between low-LET dose-response relations and the underlying kinetics of damage production/repair/misrepair. Int J Radiat Biol 72:351-374 (1997).

Schnarr K, Dayes I, Sathya D, and Boreham D. Individual radiosensitivity and its relevance to health physics. Dose-Response 5:333-348 (2007).

Scott, J. G. et al. A genome-based model for adjusting radiotherapy dose (GARD): a retrospective, cohort-based study. Lancet Oncol 18, 202-211, doi:10.1016/S1470-2045(16)30648-9 (2017).

Shannon, P. et al. Cytoscape: a software environment for integrated models of biomolecular interaction networks. Genome Res 13, 2498-2504, doi:10.1101/gr.1239303 (2003).

Shen, Y. J. et al. Genome-derived cytosolic DNA mediates type I interferon-dependent rejection of B cell lymphoma cells. Cell Rep 11, 460-473, doi:10.1016/j.celrep.2015.03.041 (2015).

Sjostrom, M. et al. Identification and validation of single-sample breast cancer radiosensitivity gene expression predictors. Breast Cancer Res 20, 64,doi:10.1186/s13058-018-0978-y (2018).

Sorlie, T. et al. Gene expression patterns of breast carcinomas distinguish tumor subclasses with clinical implications. Proc Natl Acad Sci U S A 98, 10869-10874, doi:10.1073/pnas.191367098 (2001).

Stanton, S. E., Adams, S. & Disis, M. L. Variation in the Incidence and Magnitude of Tumor-Infiltrating Lymphocytes in Breast Cancer Subtypes: A Systematic Review. JAMA Oncol 2, 1354-1360, doi:10.1001/jamaonco 1.2016.1061 (2016).

Storey, J. D. & Tibshirani, R. Statistical significance for genomewide studies. Proc Natl Acad Sci USA 100, 9440-9445, doi:10.1073/pnas.1530509100 (2003).

Strom, T. et al. Radiosensitivity index predicts for survival with adjuvant radiation in resectable pancreatic cancer. Radiother Oncol 117, 159-164, doi:10.1016/j.radonc.2015.07.018 (2015).

Strom, T. et al. Tumour radiosensitivity is associated with immune activation in solid tumours. Eur J Cancer 84, 304-314, doi:10.1016/j.ejca.2017.08.001 (2017).

Subramanian, A. et al. Gene set enrichment analysis: a knowledge-based approach for interpreting genome-wide expression profiles. Proc Natl Acad Sci U S A 102, 15545-15550, doi:10.1073/pnas.0506580102 (2005).

Tang, L. et al. Role of metabolism in cancer cell radioresistance and radiosensitization methods. J Exp Clin Cancer Res 37, 87, doi:10.1186/s13046-018-0758-7 (2018).

Teer, J. K. et al. Evaluating somatic tumor mutation detection without matched normal samples. Hum Genomics 11, 22, doi:10.1186/s40246-017-0118-2 (2017).

Thorsson, V. et al. The Immune Landscape of Cancer. Immunity 51, 411-412, doi:10.1016/j.immuni.2019.08.004 (2019).

Torres-Roca, J. F. et al. Integration of a Radiosensitivity Molecular Signature Into the Assessment of Local Recurrence Risk in Breast Cancer. Int J Radiat Oncol Biol Phys 93, 631-638, doi:10.1016/j.ijrobp.2015.06.021 (2015).

Tubiana M. Tumor cell proliferation kinetics and tumor growth rate. Acta Oncologica 28.1: 113-121 (1989).

Wang, K., Li, M. & Hakonarson, H. ANNOVAR: functional annotation of genetic variants from high-throughput sequencing data. Nucleic Acids Res 38, e164, doi:10.1093/nar/gkq603 (2010).

Weichselbaum R R, Liang H, Deng L, Fu Y.-X. Radiotherapy and immunotherapy: a beneficial liaison? Nature Reviews Clin Oncol 14: 365-379 (2017).

Wellenstein, M. D. & de Visser, K. E. Cancer-Cell-Intrinsic Mechanisms Shaping theTumor Immune Landscape. Immunity 48, 399-416, doi:10.1016/j.immuni.2018.03.004 (2018).

Welsh, E. A., Eschrich, S. A., Berglund, A. E. & Fenstermacher, D. A. Iterative rank-order normalization of gene expression microarray data. BMC Bioinformatics 14, 153, doi:10.1186/1471-2105-14-153 (2013).

Yard, B. D. et al. A genetic basis for the variation in the vulnerability of cancer to DNA damage. Nat Commun 7, 11428, doi:10.1038/ncomms11428 (2016).

Yoshihara, K. et al. Inferring tumour purity and stromal and immune cell admixture from expression data. Nat Commun 4, 2612, doi:10.1038/ncomms3612 (2013).

Zhao, S. G. et al. The Immune Landscape of Prostate Cancer and Nomination of PD-L2 as a Potential Therapeutic Target. J Natl Cancer Inst 111, 301-310, doi:10.1093/jnci/djyl41 (2019).

Zitvogel, L., Galluzzi, L., Kepp, O., Smyth, M. J. & Kroemer, G. Type I interferons in anticancer immunity. Nat Rev Immunol 15, 405-414, doi:10.1038/nri3845 (2015).

1. A method of detecting the radiosensitivity of a tumor in a subject comprising obtaining a tumor tissue sample, measuring the individual radiation immune sensitivity (iRIS) phenotype of the tumor tissue sample by measuring the pre-radiation distance of tumor-immune ecosystem (TIES) to the immune mediated tumor elimination (IMTE) region, and the number of effector, suppressor and cancer cells; wherein a iRIS value lower than a median iRIS score) (iRIS^{lo} indicates that a tumor is radiosensitive and an iRIS value above the median iRIS score (iRIS^{hi}) indicates that a tumor is radioresistant.

2. The method of detecting the radiosensitivity of a tumor of claim 1, wherein the median iRIS score is 3.61.

3. The method of detecting the radiosensitivity of a tumor of claim 1, further comprising measuring radio sensitivity index (RSI) of the tumor tissue sample and identifying the cell populations as RSI^{lo} or RSI^{hi} based on the RSI value compared to a median RSI score;
wherein a RSI value lower than a median RSI score (RSI^{lo}) indicates that a tumor is radiosensitive and an RSI value above the median RSI score (RSI^{hi}) indicates that a tumor is radioresistant; and wherein a combined iRIS/RSI phenotype of iRIS^{lo}/RSI^{lo} indicates that the tumor is radiosensitive and wherein a combined phenotype of iRIS^{lo}/RSI^{hi}, iRIS^{hi}/RSI^{lo}, or iRIS^{hi}/RSI^{hi} indicates that eh tumor is radioresistant.

4. The method of detecting the radiosensitivity of a tumor of claim 3, wherein the median RSI score is 0.42.

5. The method of detecting the radiosensitivity of a tumor of claim 1, further comprising administering fewer fractions of a total radiation dose to a subject with a tumor that has an iRIS^{lo}/RSI^{lo} phenotype.

6. The method of detecting the radiosensitivity of a tumor of claim 5, wherein the total number of fractions administered is less than 20.

7. The method of detecting the radiosensitivity of a tumor of claim 1, further comprising administering more fractions of a total radiation dose to a subject with a tumor that has an iRIS^{lo}/RSI^{hi}, iRIS^{hi}/RSI^{lo}or iRIS^{hi}/RSI^{hi} phenotype.

8. The method of detecting the radiosensitivity of a tumor of claim 7, wherein the total number of fractions administered is greater than 20.

9. The method of detecting the radiosensitivity of a tumor of claim 1, wherein the radiation dose per fraction administered is between 1.5 and 2.5 GY.

10. A method of treating a cancer in a subject comprising obtaining a tumor tissue sample from the subject; measuring the individual radiation immune sensitivity (iRIS), wherein a tumor tissue sample from a cancer that has an iRIS score lower than a median iRIS score is designated as iRIS^{lo}; measuring the radio sensitivity index (RSI) of the tumor tissue sample, wherein a RSI value lower than a median RSI score is designated as RSI^{lo}; and administering to the subject a total dosage of radiation; wherein the total dosage of radiation is administered over several fractions; and wherein the total number of fractions administered is less than 20 when the iRIS/RSI phenotype is iRIS^{lo}/RSI^{lo}; and wherein the total number of fractions administered is greater than 20 when the iRIS/RSI phenotype is iRIS^{lo}/RSI^{hi}, iRIS^{hi}/RSI^{lo}, or iRIS^{hi}/RSI^{hi}.

11. The method of treating a cancer of claim 10, wherein the administered dose of radiation is between 1.5 and 2.5 GY per fraction.
